SEQUENCE LISTING (13 sequences)

SEQ ID NO: 1 - protein, 11 aa, Influenza A virus
GLFGAIAGFIE

SEQ ID NO: 2 - protein, 4 aa, Artificial Sequence
  peptide linker sequence
GGGG

SEQ ID NO: 3 - DNA, 927 bp, Artificial Sequence
  H1 HA stem encoding construct
  CDS (1)..(927)
atgcccatggggtctctgcaaccgctggccaccttgtacctgctggggatgctggtcgcttccgtgctagctgacaccatttgtattggataccacgctaataactctaccgataccgtcgataccgtcctggaaaagaatgtcacagtcacccactctgtgaacctgctcgaagacggaggaggcggcaagtacgtgtgcagcaccaagctgaggatggctaccggcctgaggaacaagccctccaagcagagccagggcctgttcggagctatcgctggcttcaccgagggaggatggaccggaatgatcgacggatggtacggctaccaccaccagaacgagcagggatccggatacgctgccgaccagaagagcacccagaacgccatcgacggcatcaccaacaaggtgaactccgtgatcgagaagatgaacacccagtacaccgccatcggctgcgagtacaacaagagcgagaagtgcatgaagcagatcgaggacaagatcgaggagatcgagtccaaagtgtggtgctacaacgccgagctgctggtgctgctggagaacgagaggaccctggacttccacgactccaacgtgaagaacctgtacgagaaggtgaagagccagctgaagaacaacgccaaggagatcggcaacggctgcttcgagttctaccacaagtgcgacaacgagtgcatggagagcgtgaagaacggcacctacgactacccaaagtactccgaggagagcaagctgaaccgcgagaagatcgacggcgtgaagctcgagtccatgggcgtgtaccagatcctggccatctacagcaccgtggcctccagcctggtgctgctggtgtccctcggagctatttctttttggatgtgcagcaatgggtctctccagtgtaggatttgtatttga

SEQ ID NO: 4 - protein, 308 aa, Artificial Sequence
  Synthetic Construct
MPMGSLQPLATLYLLGMLVASVLADTICIGYHANNSTDTVDTVLEKNVTVTHSVNLLEDGGGGKYVCSTKLRMATGLRNKPSKQSQGLFGAIAGFTEGGWTGMIDGWYGYHHQNEQGSGYAADQKSTQNAIDGITNKVNSVIEKMNTQYTAIGCEYNKSEKCMKQIEDKIEEIESKVWCYNAELLVLLENERTLDFHDSNVKNLYEKVKSQLKNNAKEIGNGCFEFYHKCDNECMESVKNGTYDYPKYSEESKLNREKIDGVKLESMGVYQILAIYSTVASSLVLLVSLGAISFWMCSNGSLQCRICI

SEQ ID NO: 5 - DNA, 954 bp, Artificial Sequence
  H3 HA stem encoding construct
  CDS (1)..(954)
atgcccatgggcagcctgcagcccctggccaccctgtacctgctgggcatgctggtggccagcgtgctggcccaggatttccccggcaagggcaacaacaccgccaccctgtgcctgggccaccacgccgtgcccaacggcaccctggtgaagaccatcaccgatgatcagatcgaggtgaccaacgccaccgagctggtgcagaacggcggcggcggccactacatctgccagaacaccctgaagctggccaccggcatgagaaacaagcccgagaagcagacccagggcatcttcggcgccatcgccggcttcaccgagaacggctgggagggcatggtgaacggctggtacggcttcagacaccagaacagcgagggcatcggccaggccgccgatctgaagagcacccaggccgccatcaaccagatcaacggcaagctgaacagagtgatcgagaagaccaacgagaagtaccaccagatcgagtgcgagtacaacgagagcgagggctgcatgaagcagatcgaggataagatcgaggagatcgagagcaagctgtggagctacaacgccgagctgctggtggccctggagaaccagcacaccatcgatctgaccgatagcgagatgaacaagctgttcgagaagaccagaaagcagctgagagagaacgccgaggatatgggcaacggctgcttcaagatctaccacaagtgcgataacagctgcatggagagcatcagaaacggcacctacgatcacaacgagtacagagatgaggccgtgaacaacagattccagatcaagagcgtgctggagaagagcggctacaaggattggatcctgtggatcagcttcgccatcagctgcttcctgctgtgcgccatctggatgggcttcatcatctgggcctgccagaagggcaacatcagatgcaacatctgcatctga

SEQ ID NO: 6 - protein, 317 aa, Artificial Sequence
  Synthetic Construct
MPMGSLQPLATLYLLGMLVASVLAQDFPGKGNNTATLCLGHHAVPNGTLVKTITDDQIEVTNATELVQNGGGGHYICQNTLKLATGMRNKPEKQTQGIFGAIAGFTENGWEGMVNGWYGFRHQNSEGIGQAADLKSTQAAINQINGKLNRVIEKTNEKYHQIECEYNESEGCMKQIEDKIEEIESKLWSYNAELLVALENQHTIDLTDSEMNKLFEKTRKQLRENAEDMGNGCFKIYHKCDNSCMESIRNGTYDHNEYRDEAVNNRFQIKSVLEKSGYKDWILWISFAISCFLLCAIWMGFIIWACQKGNIRCNICI

SEQ ID NO: 7 - DNA, 927 bp, Artificial Sequence
  H5 HA stem encoding construct
  CDS (1)..(927)
atgcccatggggtctctgcaaccgctggccaccttgtacctgctggggatgctggtcgcttccgtgctagcagaccagatttgtatcggctatcatgccaacaactcaacagaacaggtggacactattatggagaagaatgtgacagtgacccacgcacaggacatcctggagaagggaggaggcggcaagtacgtgtgctctaaccggctggtgctggccaccggcctgagaaataagccccagaaggagacacagggcctgttcggagcaatcgcaggctttaccgagggaggatggcagggaatggtggatggatggtacggctatcaccactctaacgagcagggcagcggctatgccgccgacaaggagtctacccagaaggccatcgatggcgtgacaaacaaggtgaatagcatcatcgacaagatgaatacccagtacgaggccatcggctgcgagtataacaattccgagaggtgtatgaagcagatcgaggataagatcgaggagatcgagtctaaagtgtggtgctacaacgccgagctgctggtgctgatggagaatgagcgcacactggacttccacgatagcaacgtgaagaatctgtatgacaaggtgaggctgcagctgcgcgataacgccaaggagctgggcaatggctgtttcgagttttaccacaagtgcgacaacgagtgtatggagagcgtgcggaatggcacctacgattatcctcagtattccgaggaggcccggctgaagagagaggagatctccggcgtgaagctggagtctatcggcatctaccagatcctgtccatctattccacagtggccagctccctggccctggccatcatggtggctgggctgtccctgtggatgtgctctaacgggtccctgcagtgtcgcatttgtatttga

SEQ ID NO: 8 - protein, 308 aa, Artificial Sequence
  Synthetic Construct
MPMGSLQPLATLYLLGMLVASVLADQICIGYHANNSTEQVDTIMEKNVTVTHAQDILEKGGGGKYVCSNRLVLATGLRNKPQKETQGLFGAIAGFTEGGWQGMVDGWYGYHHSNEQGSGYAADKESTQKAIDGVTNKVNSIIDKMNTQYEAIGCEYNNSERCMKQIEDKIEEIESKVWCYNAELLVLMENERTLDFHDSNVKNLYDKVRLQLRDNAKELGNGCFEFYHKCDNECMESVRNGTYDYPQYSEEARLKREEISGVKLESIGIYQILSIYSTVASSLALAIMVAGLSLWMCSNGSLQCRICI

SEQ ID NO: 9 - DNA, 927 bp, Artificial Sequence
  H7 HA stem encoding construct
  CDS (1)..(927)
atgcccatggggtctctgcaaccgctggccaccttgtacctgctggggatgctggtcgcttccgtgctagcagacaaaatctgcctgggacatcatgccgtgagcaatggaaccaaagtgaataccctgaccgaaagaggagtggaagtcgtgaacgcaactgagacagtggagaggggaggaggcggccggtacgtgtgccagcggagcctgctgctggcaacaggcatgaagaacaagcccgagaagcctaagggacagggcctgttcggagcaatcgcaggctttaccgagaacggctgggagggcctgatcgatggctggtacggcttccggcaccagaatgcacagggagagggcaccgccgccgattataagagcacacagtccgccatcgaccagatcaccggcaagctgaaccggctgatcgagaagacaaatcagcagtacgagctgatcgattgcgagtataacgagagcgagaagtgtatgaagcagatcgaggacaagatcgaggagatcgagtccaaagtgtggtgctacaacgccgagctgctggtggctatggagaatcagcacacaatcgatctggccgactccgagatggataagctgtatgagcgggtgaagagacagctgagggagaatgccgaggaggacggcaccggctgtttcgagattttccacaagtgcgacgatgactgtatggcctccatcagaaacaatacatacgaccactctaagtatagggaggaggccatgcagaaccgcatccagatcgatcccgtgaagttaattaacggctataaggacgtgatcctgtggttctcttttggcgccagctgttttatcctgctggccatcgtgatgggactggtgtttatttgcgtcaagaacgggaacatgaggtgtactatttgtatttga

SEQ ID NO: 10 - protein, 308 aa, Artificial Sequence
  Synthetic Construct
MPMGSLQPLATLYLLGMLVASVLADKICLGHHAVSNGTKVNTLTERGVEVVNATETVERGGGGRYVCQRSLLLATGMKNKPEKPKGQGLFGAIAGFTENGWEGLIDGWYGFRHQNAQGEGTAADYKSTQSAIDQITGKLNRLIEKTNQQYELIDCEYNESEKCMKQIEDKIEEIESKVWCYNAELLVAMENQHTIDLADSEMDKLYERVKRQLRENAEEDGTGCFEIFHKCDDDCMASIRNNTYDHSKYREEAMQNRIQIDPVKLINGYKDVILWFSFGASCFILLAIVMGLVFICVKNGNMRCTICI

SEQ ID NO: 11 - DNA, 927 bp, Artificial Sequence
  H9 HA stem encoding construct
  CDS (1)..(927)
atgcccatggggtctctgcaaccgctggccaccttgtacctgctggggatgctggtcgcttccgtgctagcagataaaatctgtattggacatcagtcaactaactctaccgaaaccgtggacactctgaccgaaactaacgtgcccgtgacacatgccaaagagctgctgcacactggcggagggggcaagtacatctgcgtgaagagcctgaaactggcaattgggctgcgcaacaagcccgccaaaagctcccagggactgttcggggcaatcgccggatttactgagggagggtggcctgggctggtggcaggctggtacggattccagcactccaacgatcagggcgtgggaatggccgctgatagggacagcacacagaaggctgtggacaaaatcacttccaaggtgaacaatattgtggataagatgaacaaacagtacgagatcattgactgcgaatacaatgagagcgaaacctgcatgaaacagatcgaggataagatcgaggaaatcgaatccaagatctggtgctacaacgctgagctgctggtgctgctggaaaatcagaagacactggatgagcacgacgcaaacgtgaacaatctgtacaataaggtgaaaagagctctgggcagcaacgcaatggaggacgggaaaggctgctttgaactgtaccacaagtgcgatgaccagtgcatggaaactatcaggaacggaacctacaatagaaggaaataccaggaagagtcccgcctggagcggcagaaaattgaaggggtgaagctcgagagcgaaggcacctacaagatcctgaccatttactccacagtggccagctccctggtgctggctatgggattcgctgccttcctgttttgggctatgagcaatgggagttgtaggtgtaacatctgtatctga

SEQ ID NO: 12 - protein, 308 aa, Artificial Sequence
  Synthetic Construct
MPMGSLQPLATLYLLGMLVASVLADKICIGHQSTNSTETVDTLTETNVPVTHAKELLHTGGGGKYICVKSLKLAIGLRNKPAKSSQGLFGAIAGFTEGGWPGLVAGWYGFQHSNDQGVGMAADRDSTQKAVDKITSKVNNIVDKMNKQYEIIDCEYNESETCMKQIEDKIEEIESKIWCYNAELLVLLENQKTLDEHDANVNNLYNKVKRALGSNAMEDGKGCFELYHKCDDQCMETIRNGTYNRRKYQEESRLERQKIEGVKLESEGTYKILTIYSTVASSLVLAMGFAAFLFWAMSNGSCRCNICI

SEQ ID NO: 13 - DNA, 10705 bp, Artificial Sequence
  DNA plasmid for expression of VEEV replicon RNA
gactcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctctctggctaactagtaatacgactcactatagcgccgcctatctgatgagtccgtgaggacgaaactataggaaaggaattcctatagtcataggcggcgcatgagagaagcccagaccaattacctacccaaaatggagaaagttcacgttgacatcgaggaagacagcccattcctcagagctttgcagcggagcttcccgcagtttgaggtagaagccaagcaggtcactgataatgaccatgctaatgccagagcgttttcgcatctggcttcaaaactgatcgaaacggaggtggacccatccgacacgatccttgacattggaagtgcgcccgcccgcagaatgtattctaagcacaagtatcattgtatctgtccgatgagatgtgcggaagatccggacagattgtataagtatgcaactaagctgaagaaaaactgtaaggaaataactgataaggaattggacaagaaaatgaaggaactcgccgccgtcatgagcgaccctgacctggaaactgagactatgtgcctccacgacgacgagtcgtgtcgctacgaagggcaagtcgctgtttaccaggatgtatacgcggttgacggaccgacaagtctctatcaccaagccaataagggagttagagtcgcctactggataggctttgacaccaccccttttatgtttaagaacttggctggagcatatccatcatactctaccaactgggccgacgaaaccgtgttaacggctcgtaacataggcctatgcagctctgacgttatggagcggtcacgtagagggatgtccattcttagaaagaagtatttgaaaccatccaacaatgttctattctctgttggctcgaccatctaccacgagaagagggacttactgaggagctggcacctgccgtctgtatttcacttacgtggcaagcaaaattacacctgccgctgcgagaccatcgtgtcctgcgacggctacgtggtgaagcgcatcgccatctcccccggcctgtacggcaagccctccggctacgccgccaccatgcaccgcgagggcttcctgtgctgcaaggtgacagacaccctgaacggcgagcgcgtgtccttccccgtgtgcacctacgtgcccgccaccctgtgcgaccagatgaccggcatcctggccaccgacgtgtccgccgacgacgcccagaagctgctggtgggcctgaaccagcgcatcgtggtgaacggtcgcacccagagaaacaccaataccatgaaaaattaccttttgcccgtagtggcccaggcatttgctaggtgggcaaaggaatataaggaagatcaagaagatgaaaggccactaggactacgagatagacagttagtcatggggtgttgttgggcttttagaaggcacaagataacatctatttataagcgcccggatacccaaaccatcatcaaagtgaacagcgatttccactcattcgtgctgcccaggataggcagtaacacattggagatcgggctgagaacaagaatcaggaaaatgttagaggagcacaaggagccgtcacctctcattaccgccgaggacgtacaagaagctaagtgcgcagccgatgaggctaaggaggtgcgtgaagccgaggagttgcgcgcagctctaccacctttggcagctgatgttgaggagcccactctggaggcagacgtcgacttgatgttacaagaggctggggccggctcagtggagacacctcgtggcttgataaaggttaccagctacgatggcgaggacaagatcggctcttacgctgtgctttctccgcaggctgtactcaagagtgaaaaattatcttgcatccaccctctcgctgaacaagtcatagtgataacacactctggccgaaaagggcgttatgccgtggaaccataccatggtaaagtagtggtgccagagggacatgcaatacccgtccaggactttcaagctctgagtgaaagtgccaccattgtgtacaacgaacgtgagttcgtaaacaggtacctgcaccatattgccacacatggaggagcgctgaacactgatgaagaatattacaaaactgtcaagcccagcgagcacgacggcgaatacctgtacgacatcgacaggaaacagtgcgtcaagaaagaactagtcactgggctagggctcacaggcgagctggtggatcctcccttccatgaattcgcctacgagagtctgagaacacgaccagccgctccttaccaagtaccaaccataggggtgtatggcgtgccaggatcaggcaagtctggcatcattaaaagcgcagtcaccaaaaaagatctagtggtgagcgccaagaaagaaaactgtgcagaaattataagggacgtcaagaaaatgaaagggctggacgtcaatgccagaactgtggactcagtgctcttgaatggatgcaaacaccccgtagagaccctgtatattgacgaagcttttgcttgtcatgcaggtactctcagagcgctcatagccattataagacctaaaaaggcagtgctctgcggggatcccaaacagtgcggtttttttaacatgatgtgcctgaaagtgcattttaaccacgagatttgcacacaagtcttccacaaaagcatctctcgccgttgcactaaatctgtgacttcggtcgtctcaaccttgttttacgacaaaaaaatgagaacgacgaatccgaaagagactaagattgtgattgacactaccggcagtaccaaacctaagcaggacgatctcattctcacttgtttcagagggtgggtgaagcagttgcaaatagattacaaaggcaacgaaataatgacggcagctgcctctcaagggctgacccgtaaaggtgtgtatgccgttcggtacaaggtgaatgaaaatcctctgtacgcacccacctcagaacatgtgaacgtcctactgacccgcacggaggaccgcatcgtgtggaaaacactagccggcgacccatggataaaaacactgactgccaagtaccctgggaatttcactgccacgatagaggagtggcaagcagagcatgatgccatcatgaggcacatcttggagagaccggaccctaccgacgtcttccagaataaggcaaacgtgtgttgggccaaggctttagtgccggtgctgaagaccgctggcatagacatgaccactgaacaatggaacactgtggattattttgaaacggacaaagctcactcagcagagatagtattgaaccaactatgcgtgaggttctttggactcgatctggactccggtctattttctgcacccactgttccgttatccattaggaataatcactgggataactccccgtcgcctaacatgtacgggctgaataaagaagtggtccgtcagctctctcgcaggtacccacaactgcctcgggcagttgccactggaagagtctatgacatgaacactggtacactgcgcaattatgatccgcgcataaacctagtacctgtaaacagaagactgcctcatgctttagtcctccaccataatgaacacccacagagtgacttttcttcattcgtcagcaaattgaagggcagaactgtcctggtggtcggggaaaagttgtccgtcccaggcaaaatggttgactggttgtcagaccggcctgaggctaccttcagagctcggctggatttaggcatcccaggtgatgtgcccaaatatgacataatatttgttaatgtgaggaccccatataaataccatcactatcagcagtgtgaagaccatgccattaagcttagcatgttgaccaagaaagcttgtctgcatctgaatcccggcggaacctgtgtcagcataggttatggttacgctgacagggccagcgaaagcatcattggtgctatagcgcggcagttcaagttttcccgggtatgcaaaccgaaatcctcacttgaagagacggaagttctgtttgtattcattgggtacgatcgcaaggcccgtacgcacaatccttacaagctttcatcaaccttgaccaacatttatacaggttccagactccacgaagccggatgtgcaccctcatatcatgtggtgcgaggggatattgccacggccaccgaaggagtgattataaatgctgctaacagcaaaggacaacctggcggaggggtgtgcggagcgctgtataagaaattcccggaaagcttcgatttacagccgatcgaagtaggaaaagcgcgactggtcaaaggtgcagctaaacatatcattcatgccgtaggaccaaacttcaacaaagtttcggaggttgaaggtgacaaacagttggcagaggcttatgagtccatcgctaagattgtcaacgataacaattacaagtcagtagcgattccactgttgtccaccggcatcttttccgggaacaaagatcgactaacccaatcattgaaccatttgctgacagctttagacaccactgatgcagatgtagccatatactgcagggacaagaaatgggaaatgactctcaaggaagcagtggctaggagagaagcagtggaggagatatgcatatccgacgactcttcagtgacagaacctgatgcagagctggtgagggtgcatccgaagagttctttggctggaaggaagggctacagcacaagcgatggcaaaactttctcatatttggaagggaccaagtttcaccaggcggccaaggatatagcagaaattaatgccatgtggcccgttgcaacggaggccaatgagcaggtatgcatgtatatcctcggagaaagcatgagcagtattaggtcgaaatgccccgtcgaagagtcggaagcctccacaccacctagcacgctgccttgcttgtgcatccatgccatgactccagaaagagtacagcgcctaaaagcctcacgtccagaacaaattactgtgtgctcatcctttccattgccgaagtatagaatcactggtgtgcagaagatccaatgctcccagcctatattgttctcaccgaaagtgcctgcgtatattcatccaaggaagtatctcgtggaaacaccaccggtagacgagactccggagccatcggcagagaaccaatccacagaggggacacctgaacaaccaccacttataaccgaggatgagaccaggactagaacgcctgagccgatcatcatcgaagaggaagaagaggatagcataagtttgctgtcagatggcccgacccaccaggtgctgcaagtcgaggcagacattcacgggccgccctctgtatctagctcatcctggtccattcctcatgcatccgactttgatgtggacagtttatccatacttgacaccctggagggagctagcgtgaccagcggggcaacgtcagccgagactaactcttacttcgcaaagagtatggagtttctggcgcgaccggtgcctgcgcctcgaacagtattcaggaaccctccacatcccgctccgcgcacaagaacaccgtcacttgcacccagcagggcctgctcgagaaccagcctagtttccaccccgccaggcgtgaatagggtgatcactagagaggagctcgaggcgcttaccccgtcacgcactcctagcaggtcggtctcgagaaccagcctggtctccaacccgccaggcgtaaatagggtgattacaagagaggagtttgaggcgttcgtagcacaacaacaatgacggtttgatgcgggtgcatacatcttttcctccgacaccggtcaagggcatttacaacaaaaatcagtaaggcaaacggtgctatccgaagtggtgttggagaggaccgaattggagatttcgtatgccccgcgcctcgaccaagaaaaagaagaattactacgcaagaaattacagttaaatcccacacctgctaacagaagcagataccagtccaggaaggtggagaacatgaaagccataacagctagacgtattctgcaaggcctagggcattatttgaaggcagaaggaaaagtggagtgctaccgaaccctgcatcctgttcctttgtattcatctagtgtgaaccgtgccttttcaagccccaaggtcgcagtggaagcctgtaacgccatgttgaaagagaactttccgactgtggcttcttactgtattattccagagtacgatgcctatttggacatggttgacggagcttcatgctgcttagacactgccagtttttgccctgcaaagctgcgcagctttccaaagaaacactcctatttggaacccacaatacgatcggcagtgccttcagcgatccagaacacgctccagaacgtcctggcagctgccacaaaaagaaattgcaatgtcacgcaaatgagagaattgcccgtattggattcggcggcctttaatgtggaatgcttcaagaaatatgcgtgtaataatgaatattgggaaacgtttaaagaaaaccccatcaggcttactgaagaaaacgtggtaaattacattaccaaattaaaaggaccaaaagctgctgctctttttgcgaagacacataatttgaatatgttgcaggacataccaatggacaggtttgtaatggacttaaagagagacgtgaaagtgactccaggaacaaaacatactgaagaacggcccaaggtacaggtgatccaggctgccgatccgctagcaacagcgtatctgtgcggaatccaccgagagctggttaggagattaaatgcggtcctgcttccgaacattcatacactgtttgatatgtcggctgaagactttgacgctattatagccgagcacttccagcctggggattgtgttctggaaactgacatcgcgtcgtttgataaaagtgaggacgacgccatggctctgaccgcgttaatgattctggaagacttaggtgtggacgcagagctgttgacgctgattgaggcggctttcggcgaaatttcatcaatacatttgcccactaaaactaaatttaaattcggagccatgatgaaatctggaatgttcctcacactgtttgtgaacacagtcattaacattgtaatcgcaagcagagtgttgagagaacggctaaccggatcaccatgtgcagcattcattggagatgacaatatcgtgaaaggagtcaaatcggacaaattaatggcagacaggtgcgccacctggttgaatatggaagtcaagattatagatgctgtggtgggcgagaaagcgccttatttctgtggagggtttattttgtgtgactccgtgaccggcacagcgtgccgtgtggcagaccccctaaaaaggctgtttaagcttggcaaacctctggcagcagacgatgaacatgatgatgacaggagaagggcattgcatgaagagtcaacacgctggaaccgagtgggtattctttcagagctgtgcaaggcagtagaatcaaggtatgaaaccgtaggaacttccatcatagttatggccatgactactctagctagcagtgttaaatcattcagctacctgagaggggcccctataactctctacggctaacctgaatggactacgacatagtctagtccgccaagatatcttcagggcgcgccgcaccatgtgattaattaagtaacgatacagcagcaattggcaagctgcttacatagaactcgcggcgattggcatgccgctttaaaatttttattttatttttcttttcttttccgaatcggattttgtttttaatatttcaaaaaaaaaaaaaaaaaaaaaaaaaaaaaaaaaaaaaaaaaaaaaaggaagagcgcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattattaacgcttacaatttcctgatgcggtattttctccttacgcatctgtgcggtatttcacaccgcatacaggtggcacttttcggggaaatgtgcgcggaacccctatttgtttatttttctaaatacattcaaatatgtatccgctcatgagacaataaccctgataaatgcttcaataatagcacgtgctaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctgggcttttgctggccttttgctcacatgttctt

(19) United States
(12) Patent Application Publication
(10) Pub. No.: US 2024/0024456 A1
(43) Pub. Date: Jan. 25, 2024

(54) HA STEM VACCINE FOR HA ANTIBODY-POSITIVE TARGETS

(71) Applicant: Intervet Inc., Madison, NJ (US)

(72) Inventors: Martijn Alexander Langereis, Boxmeer (NL); Iwan Verstegen, Boxmeer (NL); Maria Alexander Cornelis De Haan, Utrecht (NL)

(73) Assignee: Intervet Inc., Madison, NJ (US)

(21) Appl. No.: 18/043,944

(22) PCT Filed: Sep. 6, 2021
(86) PCT No.: PCT/EP2021/074446

§ 371 Date: Mar. 3, 2023

(30) Foreign Application Priority Data
Sep. 7, 2020 (EP) 20194937.7

(51) Int. Cl. A61K 39/145; C07K 14/11; C12N 15/86; A61P 31/16

(52) U.S. Cl. CPC A61K 39/145; C07K 14/11; C12N 15/86; A61P 31/16

(57) ABSTRACT

The present invention relates to vaccines against influenza virus infection or disease for targets with pre-existing antibodies against influenza virus HA head domain. The invention regards a recombinant vector expressing a HA stem polypeptide, a vaccine comprising the vector or a host cell with said vector, uses of the vector, the host cell, or the vaccine, and methods for reducing influenza virus infection or disease. The recombinant vector can be a nucleic acid such as a eukaryotic expression plasmid or an RNA, a virus, or a replicon particle (RP). This vaccination allows for the induction of an early- and effective immune-response against Influenza virus induced infection or disease, not hindered by pre-existing anti-HA head domain antibodies.

The present invention relates to the field of vaccinology, specifically to vaccines against influenza. In particular the invention relates to vaccines against influenza virus infection or disease for targets with pre-existing antibodies against influenza virus HA protein. The invention regards a recombinant vector expressing a HA stem polypeptide, a vaccine comprising the vector or a host cell with said vector, uses of the vector, the host cell, or the vaccine, and methods for reducing influenza virus infection or disease.

Influenza virus occurs worldwide and infects both humans and animals. Disease caused is mainly respiratory with a variety of additional symptoms. Pathology varies from mild to lethal, leading to much discomfort and economic damage. Further there is a chance of the zoonotic infection of humans by infected birds or porcines.

Influenza virus is an enveloped virus from the Orthomyxoviridae family and has a single-stranded, negative-sense, RNA genome that is segmented. Influenza virus A-D are separate genera within that family, differentiated based on their structural proteins (matrix and nucleoprotein). Most prominent worldwide are Influenza A, B, and C viruses. Of these, the genus Influenza A covers several strains of the virus that infect only a specific target species, but also some that have a multi-species host range. Serological subdivision is made on the basis of the viral envelope glycoproteins expressed: haemagglutinin (HA) and neuraminidase (NA). Currently 18 different HA antigens are known, indicated as H1 through H18, and 11 NA antigens: N1-N11. Details on Influenza virus and of the diseases it can induce are described in well-known handbooks such as: Fields Virology (LWW publ., ISBN: 9781451105636); The Merck veterinary manual (2010, 10th ed., C. M. Kahn edt., ISBN: 091191093X); and: Diseases of poultry (2008, 12th ed., Y. Saif ed., Iowa State Univ. press, ISBN-10: 0813807182). Influenza infection in poultry is also called: fowl plague, avian flu, or bird flu.

The HA protein is the main antigen of influenza virus A and B, and is central to the virus' recognition-, binding-, and entry of a host cell. In its natural form the HA protein is a homo-trimer that is displayed on the envelope (or ‘coat’) of the virus and the membrane of an infected host cell. Each monomer has a globular head domain that is connected via a stem domain to a transmembrane domain. The head domain provides the trimerization, and mediates the initial contact with the host cell receptor. The stem (or stalk) domain of HA, after endocytosis of the virus, induces fusion with the host cell's endosomal membrane.

To prevent pre-mature membrane fusions, in nature the HA protein is expressed as an inactive pre-protein HA0 (HA zero), which is post-translationally cleaved by a host protease into HA1 and HA2 sections. The central part of HA1 forms the head domain of a HA monomer. The main part of HA2 forms the stem domain, together with N- and C-terminal parts of HA1. Further HA2 contains at its C-terminus the transmembrane domain and a short cytoplasmic domain.

Of an HA antigen, the head domain is the immunodominant part. Consequently, when a target is immunised with a preparation comprising a HA head domain or an antigenic part thereof, such as full length HA protein, or even a whole Influenza virus preparation, antibodies generated in the target are mainly directed at the HA head domain. Because the part of the HA gene encoding the head domain is variable in sequence, this explains the bulk of the antigenic drift displayed by (seasonal) variants of influenza virus. Consequently there is a constant need for new and updated vaccines against influenza virus, both for human- and for animal targets.

Influenza vaccination is routinely applied both in human- and in veterinary medical practice. Goal is typically to reduce severity and duration of clinical signs, and preferably also to reduce the amount and duration of viral shedding by the infected host. Several different types of vaccines are available, e.g. based on live attenuated virus, or on adjuvated formulations of inactivated virus, of virus preparations such as detergent-extract (so-called ‘split’) vaccine, or of subunit vaccines of HA and/or NA proteins. Also vaccines may be based on a recombinant product such as e.g. expression plasmids, mRNA, a vector virus, or virus-like particles.

In veterinary practice, influenza vaccines are available for a variety of animal species. Such vaccination may be applied incidentally to e.g. poultry or swine as an emergency vaccination in outbreak situations. Alternatively, in countries where infection-pressure by influenza virus is high, vaccination may be routinely applied to swine- and poultry, and to their offspring at very young age.

The vaccinated target human or animal may contain (maternally derived) antibodies against influenza resulting from a prior contact with the virus from a vaccination or a field-infection, either of the target itself, or of their mother. Such pre-existing antibodies in the target are known to interfere with the efficacy of Influenza vaccination. This severely reduces the efficacy of Influenza vaccination of such antibody-positive targets, leaving them exposed to field infections. No effective solution has been provided for this problem so far.

Contrary to the HA head domain, the stem domain of HA is more conserved among influenza virus strains, and can be used to induce broad virus-recognising antibodies when administered without HA head domain (i.e. ‘headless’). This was recognised already in 1993 (Okuno et al., 1993, J. of Virol., vol. 67, p. 2552-2558). Since then many studies have used a so-called ‘headless HA’, ‘mini-HA’, or ‘HA stem’ polypeptide as vaccine antigen, in an attempt to provide a universal influenza vaccine. This is e.g. reviewed by Krammer & Palese (2013, Curr. Opin. Virol., vol. 3, p. 521-530), and: by Ostrowsky et al. (2020, Curr. Opin. Virol., vol. 40, p. 28-36).

Many details are known on the structure of the Influenza HA protein. Reviews are e.g.: Skehel & Wiley (2000, Annu. Rev. Biochem., vol. 69, p. 531-569); Sriwilaijaroen & Suzuki (2012, Proc. Jpn. Acad., Ser. B, vol. 88, p. 226-249); and Russell (2016, Ref. Module in Biomed. Sci., doi:10.1016/B978-0-12-801238-3.95721-0). An illustrative graphical representation of the various domains and segments of the Influenza A HA protein is provided in FIG. 1 of Lu et al. (2014, PNAS, vol. 11, p. 125-130).

WO 2011/123495 (495) describes Influenza HA stem domain polypeptides and their use as vaccines. The HA stem polypeptides comprise an HA1 N-terminal stem segment, an HA1 C-terminal stem segment and an HA2, optionally with one or more linkers and a trimerization domain. In its FIGS. 1 and 2, '495 presents illustrative multiple alignments of HA amino acid sequences, and in its Tables 1-7 illustrative amino acid sequences are described of various domains from different HA serotypes of Influenza A and B. While '495 mentions versions of HA2 with- or without a transmembrane domain, there is no enabling disclosure of such a construct in a recombinant vector, and no results are provided on any inoculation of a stem polypeptide into a target, human or animal, let alone of any vaccination-challenge experiment. '495 also does not disclose a use in a target with pre-existing anti-influenza HA antibodies, maternally derived or otherwise; in fact in '495 the use in young targets, e.g. a human child of less than 6 months of age, is specifically discouraged (see '495 paragraphs 397, 404 and 405).

WO 2013/079473 describes the use of HA stem antigens from H1 and H3 type Influenza virus, as broadly protective vaccine antigens. Several mutations were introduced into HA2 to increase stability and immunogenicity. Linker sequences and a signal sequence were used to optimise the expression construct. To provide for multimerization in the absence of a head domain, in some constructs the HA stem antigen was provided with a trimerization domain, and the transmembrane (TM) domain. Vaccinations were done in specific pathogen-free (SPF) mice, using expression plasmids, sometimes booster vaccinations were given using adjuvated soluble protein, and in some case challenge infection was applied.

The paper by Impagliazzo et al. (2015, Science, vol. 349, p. 1301-1306) followed-up the work in WO 2013/079473, by testing of soluble HA stem antigen (i.e. without TM domain) in SPF mice and in sero-negative macaques.

Sunwoo et al. (2018, Vaccines, vol. 6, p. 64) used chimeric HA proteins as vaccines in pigs having maternally derived antibodies (MDA) against Influenza virus. This in order to investigate a connection between vaccine-enhanced respiratory disease and stem-specific antibodies. The antigens tested consisted of chimeric full-length HA proteins of which the head domains were varied, but the stem domain was kept constant. These were administered to H1 HA MDA positive pigs, either as live attenuated recombinant influenza virus, as inactivated virus, or as virus-extract (split vaccine). While no stem-specific antibodies could be detected after vaccination, some level of protection against challenge-infection was obtained using a heterologous prime-boost vaccination regimen.

It is an object of the present invention to accommodate to a need in the field, and to provide influenza vaccines that are effective in targets having antibodies against an influenza virus HA head domain at the time of vaccination.

Surprisingly it was found that this object can be met, and consequently one or more disadvantages of the prior art can be overcome, by using a recombinant vector capable of expressing a polypeptide that has a headless HA stem domain with a transmembrane domain and a trimerization domain, for the vaccination of targets having antibodies against an influenza virus HA head domain.

Upon vaccinating chickens against Influenza, the inventors were disappointed to find that almost no protection was provided after a single vaccination with a soluble HA stem antigen as described by WO 2013/079473 and Impagliazzo et al. (supra). Apparently the effective and broad protection provided by an HA stem antigen as described in literature, could not readily be reproduced, not even in antibody-free animals, and when using an adjuvant. There was no indication in the literature how this could be changed.

It was only when the HA stem antigen was delivered by expression from a recombinant vector, and was provided with a transmembrane domain, and thus became expressed on the surface of host cells, that the HA stem antigen provided a good vaccine efficacy in targets with Influenza MDAs, even after a single vaccination. This has not been previously disclosed in the art.

The HA stem domain antigen with a transmembrane domain could be expressed in targets by administration of different types of the recombinant vector, e.g. of an expression plasmid, a replicon RNA, a recombinant viral vector, or as replicon particles (RPs). This provides broad opportunities for utilisation as an Influenza vaccine for targets with pre-existing antibodies against an Influenza virus HA head domain at the time of vaccination.

It is not known how or why a vector-expressed and membrane-displayed HA stem domain is so much more effective as antigen than a soluble HA stem antigen in the context of pre-existing HA antibodies. Although the inventors do not want to be bound by any theory or model that might explain these findings, they assume that the membrane anchor has an effect on the macro-molecular structure of the HA stem antigen, which in turn results in a more effective presentation of the stem antigen to the target's immune system. This allows for the induction of an early- and effective immune-response in a vaccinated target by the HA stem domain antigen, not hindered by pre-existing anti-HA head domain antibodies.

Therefore in one aspect the invention relates to a recombinant vector capable of expressing a recombinant Influenza virus haemagglutinin (HA) stem polypeptide in a target, for use in reducing infection or disease caused by influenza virus in a target that has antibodies against an Influenza virus HA head domain at the time of vaccination, characterised in that said polypeptide comprises a headless Influenza virus HA stem domain, a trimerization domain, and a transmembrane domain.

A “vector” is well-known in the field of the invention as a molecular structure that carries the genetic information (a nucleic acid sequence), for encoding a polypeptide, with appropriate signals to allow its expression under suitable conditions, such as in a host cell. For the invention ‘expression’ regards to the well-known principle of the expression of protein from genetic information by way of transcription and/or translation.

Many types and variants of such a vector are known and can be used for the invention, ranging from nucleic acid molecules like DNA or RNA, to more complex structures such as virus-like particles and replicon particles, up to replicating recombinant micro-organisms such as a virus.

Depending on the type of vector employed more or less expression signals need to be provided, either in cis (i.e. provided within the recombinant vector itself) or in trans (i.e. provided from a separate source).

A “recombinant” vector for the invention, is a vector of which the genetic constitution does not fully match with that of its native counterpart. Such a vector thus has a molecular make-up that was changed, typically by manipulation in vitro of its genetic information by way of molecular cloning, and recombinant protein expression techniques. The changes made can serve to provide for, to improve or to adapt the expression, manipulation, purification, stability and/or the immunological behaviour of the vector and/or of the protein it expresses. These, and other techniques are explained in great detail in standard text-books like Sambrook & Russell: “Molecular cloning: a laboratory manual” (2001, Cold Spring Harbour Laboratory Press; ISBN: 0879695773); Ausubel et al., in: Current Protocols in Molecular Biology (J. Wiley and Sons Inc, NY, 2003, ISBN: 047150338X); and C. Dieffenbach & G. Dveksler: “PCR primers: a laboratory manual” (CSHL Press, ISBN 0879696540); and “PCR protocols”, by: J. Bartlett and D. Stirling (Humana press, ISBN: 0896036421).

The skilled person is well equipped to select and combine the required signals into operational combinations to make the recombinant vector for use according to the invention “capable of expressing” the HA stem polypeptide for the invention under appropriate conditions. Next to elements to assist with the construction and cloning, such as restriction enzyme recognition sites or PCR primers, well-known elements can be selected from one or more of a: promoter, stop codon, termination signal, polyadenylation signal, 7-methylguanosine (7mG) cap structure, and an intron with functional splice donor- and -acceptor sites.

An “influenza virus” is well-known in the field of the invention, and such a virus has the characterising features of its taxonomic group, such as the morphologic, genomic, and biochemical characteristics, as well as the biological characteristics such as physiologic, immunologic, or pathologic behaviour.

General information on these viruses is available e.g. from reference handbooks as indicated herein. Samples of an influenza virus for use in the invention can be obtained from a variety of sources, e.g. as a field isolate from a human, or from an animal in the wild or on a farm, or from various laboratories, (depository) institutions, or (veterinary) universities. Influenza viruses can be readily identified using routine serological-, biochemical-, or molecular biological tools. In addition much sequence information on influenza viruses is available digitally in public sequence databases such as NCBI's GenBank and EMBL's EBI. In addition, detailed structural information on HA proteins is available in the Research Collaboratory for Structural Bioinformatics (RCSB) Protein Data Bank (PDB) at: www.rcsb.org, and in the Influenza Research Database at: www.fludb.org.

As is also known in the field, the classification of a micro-organism in a particular taxonomic group is based on a combination of its features. The invention therefore also includes variants of the virus' species that are sub-classified therefrom in any way, for instance as a subspecies, strain, isolate, genotype, variant, subtype or subgroup, and the like.

Further, it will be apparent to a person skilled in the field of the invention that while a particular virus for the invention may currently be assigned to this species, that is a taxonomic classification that could change in time as new insights can lead to reclassification into a new or different taxonomic group. However, as this does not change the virus itself, or its antigenic repertoire, but only it's scientific name or classification, such re-classified viruses remain within the scope of the invention.

A “haemagglutinin” (HA) (also: hemagglutinin) is a well-known envelope glycoprotein of an Influenza A or B virus, encoded by the fourth segment of the viral genome. The Influenza A HA gene encodes a preprotein of about 566 amino acids (aa) in size. Without the signal sequence, the mature HA0 protein is about 550 aa in size, with about 329 aa for the HA1, and about 221 aa for the HA2 section.

For the invention, the naming of the various domains, segments and sections of an Influenza HA protein is according to Lu et al. (supra). Further, with regard to the numbering of the amino acids in HA proteins of different subtypes, the standard uniform numbering used in this technical field is based on the so-called ‘H3 numbering’. This is because the HA of the Influenza virus isolate: A/Aichi/2/68 (H3N2) from Hong Kong in 1968, was the first to have its crystal structure fully analysed, and was later sequenced (Verhoeyen et al., 1980, Nature, vol. 286, p. 771-776). The amino acid sequence of that full-length H3 HA protein of 566 aa is represented in GenBank accession nr. AAA43178, whereby the H3-numbering system applies to the mature HA protein, thus without the 16 aa signal peptide. This approach is also the basis of the numbering scheme proposed by Burke and Smith (2014, PLoS One 9(11): e112302), which allows to identify amino acids that are structurally and functionally equivalent across all HA subtypes. The FluDB website (supra) even provides a convenient ‘HA Subtype Numbering Conversion’ tool, based on this publication by Burke and Smith. Consequently, the H3 numbering system will be used herein to identify specific amino acid residue numbers of HA polypeptides.

However when considering the size and sequence of domains, segments and sections from an HA protein, the biological variations of the HA protein from the various Influenza virus strains can be taken into consideration. For example some differences occur between HA proteins of group 1 (H1, H2, H5, H6, H8, H9, H11, H12, H13, H16, H17, and H18) and group 2 (H3, H4, H7, H10, H14, and H15). Therefore, for the size indications used herein, “about” means the size can vary by between 1 to 5 amino acids more or less, around the indicated aa residue H3 number.

A similar indication applies to Influenza B HA proteins, of which the encoded HA0 is about 585 aa in size, with about 345 aa for HA1 and about 223 for HA2.

A HA protein can be characterised in different ways, for example biochemically or serologically, all well-known to a skilled person. Further, from the prior art and common general knowledge a person skilled in the art of the invention will know and readily recognise the different domains and regions of an HA protein.

For Influenza A HA, the HA2 section starts after the Arginine amino acid at about the aa with H3 numbering 329, which is used for the proteolytic cleavage of HA0, and starts with a conserved amino acid sequence, named the ‘fusion peptide’ that contains the amino acid sequence GLFGAIAGFIE (SEQ ID NO: 1), or an aa sequence having at least 90% sequence identity with SEQ ID NO: 1; at about the aa with H3 number 330-340.

In the stem domain of the HA2 section, the region at about aa 75-90 from HA2 is the interface between natural stem trimers, this is at about the aa with H3 numbering 405-420.

At its C-terminal side, the Influenza A HA2 has a transmembrane domain of about 27 aa, in H3 numbering running from aa 514-540; and a cytoplasmic domain of about 10 aa, in H3 numbering from aa 541-550.

A “polypeptide” refers to a molecular chain of amino acids. The polypeptide can be a native or a mature protein, a pre- or pro-protein, or a fragment of a protein. Therefore proteins, peptides and oligopeptides are included within the definition of polypeptide for the invention, as long as these still contain the indicated domains of an influenza virus HA protein.

A “target” for the invention is any human or animal that is susceptible to infection by an influenza virus. The animal can for example be an avian, porcine, canine, equine, or mustelid.

The target can be of any weight, sex, or age at which it is susceptible to vaccination with the recombinant vector for use according to the invention. However it is evidently favourable to treat healthy, uninfected targets, and to treat as early as possible to prevent any field infection and its consequences.

The recombinant vector can thus be used either as a prophylactic- or as a therapeutic treatment, or both, as the polypeptide it can express, can induce an immune response that can interfere both with the establishment and with the progression of an infection with Influenza virus.

The “use” of the recombinant vector for the invention regards a human- or veterinary medical use wherein the immunologic properties of the vector-expressed polypeptide are employed. Typically this involves an immunisation with the recombinant vector as an active component in a vaccine as described herein below.

The term “reducing” regards reducing in part or in whole the establishment or the proliferation of a productive infection by an influenza virus, in cells and organs of a susceptible target, or of the subsequent signs of disease. This is achieved for example by reducing the viral load or shortening the duration of the viral replication. In turn this leads to a reduction in the target of the number, the intensity, or the severity of lesions and associated clinical signs of disease caused by the viral infection.

Such reduction of infection or disease can readily be detected for instance by monitoring the immunological response following vaccination with the recombinant vector for use according to the invention, and by testing the appearance of clinical symptoms or mortality after an infection of vaccinated targets, e.g. by monitoring the targets' signs of disease, clinical scores, serological parameters, or by re-isolation of the infecting pathogen. In animals these results can be compared to a response to a challenge infection in mock vaccinated animals. Different ways to assess influenza virus infection and symptoms of disease are well-known in the art.

The “infection or disease caused by influenza virus” refers to an infection by an influenza virus, and to the subsequent well-known symptoms of infection or disease that such infection causes, and their welfare and economic consequences.

The protection against influenza virus infection or disease induced by the expression of the HA stem polypeptide for the invention from the recombinant vector for use according to the invention, provides targets immunised with that vector, with an improvement of health and economic performance. This can for instance be assessed from parameters such as an increase of well-being, survival, growth rate, feed conversion, and production of eggs, as well as reduced costs for (veterinary) health care.

The present invention is for use in a target “which has antibodies against an Influenza virus HA head domain at the time of vaccination”. Such pre-existing antibodies interfere with an immunisation that comprises a full length influenza virus HA protein or an antigenic part of such HA head domain. However, such antibodies -as disclosed herein- do not, or at least much less, interfere with immunisation with a HA stem polypeptide of the invention.

The pre-existing antibodies directed against a HA head domain are typically acquired after infection or immunisation with an antigenic preparation that contains a HA head domain or a part thereof, as the head domain is immunodominant. Such preparation can be a whole- or a partial influenza virus preparation, live or inactivated, or a recombinant vaccine comprising a full length HA protein, a HA head domain or a part thereof.

The target can have acquired such antibodies against a HA head domain after Influenza infection or immunisation of the target itself. Alternatively, such antibodies can be obtained passively, by inoculation or ingestion of antibodies. When obtained from the target's mother, the antibodies are ‘maternally derived antibodies’ (MDA). MDA occur in several types of targets, human or animal.

In mammals MDA can derive from trans-placental transfer of antibodies from mother to off-spring. Alternatively, or additionally, MDA can be derived by ingestion of such antibodies, for example by ingestion of mother's milk containing antibodies, so-called colostrum. However, the ingestion of colostrum to obtain passive protection against disease is not exclusive for young targets, and may also be applied to older targets, and cross-species.

In avians, MDA are present in the egg yolk, which the unborn chick incorporates into its abdomen during its maturation in the egg.

The “antibodies” for the invention relate to immunoglobulins of any type: e.g. IgA, IgG, IgM, IgD, IgE, or IgY, or to parts thereof such as a single-chain variable fragment (ScFv), or Fv, F(ab′) or F(ab′)2 fragments.

Without immune-stimulation or suppletion, the level of antibodies (the so-called ‘titre’) in a target reduces over time, due to their limited biological half-life. This applies for instance to a target after birth or when the target stops to ingest milk. Therefore “at the time of vaccination” aims to connect the timing of immunisation with the recombinant vector for use according to the invention, with the titre of the anti HA head domain antibody in that target. This antibody level can e.g. be determined by taking a serum sample from the target around the time of vaccination, to determine the titre of the antibodies against the HA head domain.

For the invention, the antibody level in the target at the time of vaccination is the antibody titre in the target at a timepoint within the period of ±3 days around the time of vaccination.

However this does not preclude that the actual determination itself of the value of that pre-existing titre, i.e. the performance of the serological test on a serum sample taken around the time of vaccination, and/or the analysis and interpretation of the results of that test, can be done some considerable time after the vaccination, provided the serum sample was stored under appropriate conditions to maintain antibodies sufficiently intact, e.g. stored at −20 ° C. or colder. Similarly, this does not prevent that the pre-existing titre at the time of vaccination is calculated and extrapolated from the level determined in a sample taken some more time before- or after the vaccination, provided fairly accurate data are available on the decline of the antibodies' titre over time.

For the invention a target “has” antibodies when the titre of the anti-HA head domain antibodies in serum from that target is above a background level as detected in a comparable target human or -animal that is naïve for Influenza virus and Influenza antibodies. In animals this can e.g. be the titre as present in the serum of an SPF (specific pathogen free) animal of the same age and species.

For the invention “antibodies against” an HA head domain, refers to antibodies that specifically bind to (i.e. are specific for) a polypeptide comprising such an Influenza HA head domain, for example an HA subunit antigen or a preparation of Influenza virus. Such specificity is readily determined by a skilled person, e.g. in an ELISA, by linear dilution of the antiserum in a test using coated HA antigen. When the antibodies in the antiserum are specific, the test will display a gradual and linear decrease of the binding-signal detected.

A “HA head domain” is well-known in this field to be the central part of the HA1 section of an HA protein, which is capable of forming a 3D globular structure, and which a skilled person would know and readily recognise. When counting from a mature Influenza A HA0 protein of about 550 aa, the head domain constitutes the polypeptide comprising the amino acid sequence in H3 numbering from about aa number 44 through about aa number 274, corresponding to the indications given in Lu et al., supra. Again, variation in specific size and aa numbers may occur in different Influenza A virus HA proteins, see e.g. FIG. 1A in WO 2011/123495 for Influenza A HA sequences of serotypes H1-H16; also see Table 7 in WO2013/079473 for Influenza A H1 HA sequences.

Consequently, a “headless” HA stem domain polypeptide for the invention does not comprise the amino acid sequence corresponding to the Influenza virus HA head domain as defined above.

The term “comprises” (as well as variations such as “comprising”, “comprise”, and “comprised”) as used herein, intends to refer to all elements, and in any possible combination conceivable for the invention, that are covered by or included in the text section, paragraph, claim, etc., in which this term is used, even if such elements or combinations are not explicitly recited; and not to the exclusion of any of such element(s) or combinations.

Therefore any such text section, paragraph, claim, etc., can therefore also relate to one or more embodiment(s) wherein the term “comprises” (or its variants) is replaced by terms such as “consist of”, “consisting of”, or “consist essentially of”.

An “Influenza virus HA stem domain polypeptide” is well-known in this field to comprise two parts from the HA1 section and the main part of the HA2 section of an HA protein. A stem polypeptide can e.g. be identified by using well-known monoclonal antibodies specific for the HA stem domain of a variety of Influenza HA proteins, e.g.: FI6, CR9114 and MEDI8552, all well-known and commercially available.

Specifically, and based on the indications given in Lu et al. (supra), for a mature Influenza virus HA0 protein, a stem domain for the invention comprises:
an HA1 N-terminal stem segment, starting at the first amino acid left after cleavage of the signal sequence and running up to the start of the head domain: in H3 numbering from about aa number 1 through about aa number 43,
an HA1 C-terminal stem segment, starting after the head domain, and running up to the cleavage site of HA1 and HA2: in H3 numbering from about aa number 276 through about aa number 329, and
the HA2 ectodomain, starting after the cleavage site of HA1 and HA2, and running up to the transmembrane domain: in H3 numbering from about aa number 330 through about aa number 513.

As described in more detail below, in H3 HA the length of the HA1 N-terminal stem segment deviates more than 5 aa, and is about 10 aa longer. Consequently, for H3, this shifts the ‘H3 numbers’ for the various domains 10 aa upwards.

As the polypeptide for the invention is expressed as one fusion peptide, its constituting parts are covalently linked into one chain of amino acids, either directly, or by way of one or more intervening spacer- and/or linker amino acid sequences. Further connections may be provided by disulphide bonds formed between cysteine amino acids in the different parts.

The constituting parts of the stem domain polypeptide may be native or heterologous, whereby for the invention, a polypeptide-part is “heterologous”, if it is derived from a different source as compared to the HA2 stem domain that is in the polypeptide for the invention. Use of one or more heterologous elements creates a chimeric version of the polypeptide for the invention, which is within the scope of the invention.

For the invention, ‘derived’ indicates the origin of the polypeptide for the invention, and thus its encoding nucleic acids. These can be isolated from a biological source or can be produced recombinantly, or synthetically, on the basis of sequence information.

Changes in the polypeptide for use in the invention, respective to a native HA stem domain, regard for example: replacing the native signal sequence with a heterologous signal sequence; changing the basic amino acid(s) that signal HA1-HA2 cleavage; and/or adding a tag to facilitate purification, e.g. a 6× Histidine tag. Also, between the indicated domains that make up an Influenza virus HA stem polypeptide for the invention, one or more aa linker sequences may be used.

A “trimerization domain” is a well-known polypeptide that provides for the multimerization in tri-fold of homomer polypeptides to which it is attached. A variety of such trimerization domains are known and available in this field, for example: the isoleucine zipper 3 domain of the GCN4 transcriptional activator from Saccharomyces cerevisiae (‘GCN4 domain’), and the foldon domain of the bacteriophage T4 fibritin protein (‘foldon domain’).

The trimerization domain can be comprised by the recombinant vector for use according to the invention in different ways: e.g. before-, behind-, or in between the segments and domains that make up the recombinant vector for use according to the invention.

A “transmembrane domain” is well-known to be an amino acid sequence with hydrophobic character, which can provide for attachment to and/or anchoring in a lipid bilayer membrane. The transmembrane region that is attached to the HA stem domain polypeptide for the invention, can be the native transmembrane domain of the HA2 stem domain used in the recombinant vector for use according to the invention, or can be a heterologous transmembrane domain, either from a different Influenza virus HA protein, or from another protein.

The transmembrane domain may incorporate the Influenza virus HA protein's cytoplasmic domain, or not.

Details of embodiments and of further aspects of the invention will be described below.

In an embodiment of the recombinant vector for use according to the invention, the disease caused by influenza virus, is caused by an Influenza A virus or by an Influenza B virus; preferably the disease is caused by an Influenza A virus.

In an embodiment of the recombinant vector for use according to the invention, the Influenza virus HA stem polypeptide expressed is derived from an Influenza A virus or from an Influenza B virus; preferably the HA stem polypeptide is derived from an Influenza A virus HA protein selected from any one of the serotypes: H1 through H18; more preferably the HA stem polypeptide is derived from an Influenza A virus HA protein selected from any one of the serotypes: H1, H3, H5, H7, and H9.

In an embodiment of the recombinant vector for use according to the invention, the amino acid sequence of the HA stem polypeptide expressed, is a consensus sequence.

As is well-known, to obtain such a consensus sequence, either amino acid- or encoding nucleotide sequences are compared and a consensus sequence is derived from that comparison; for example by aligning several H9 HA stem domain nucleotide sequences using an appropriate computer program.

In an embodiment of the recombinant vector for use according to the invention, the general order of the constituting parts of the HA stem polypeptide expressed is, from N-terminal to C-terminal:
the HA1 N-terminal stem segment,
the HA1 C-terminal stem segment,
the HA2 ectodomain,
the transmembrane domain, and
the cytoplasmic domain.

In an embodiment of the recombinant vector for use according to the invention, the HA stem polypeptide expressed contains a linker sequence in between one or more of:
the HA1 N-terminal stem segment and the HA1 C-terminal stem segment;
the HA1 C-terminal stem segment and the HA2 ectodomain; and
HA2 ectodomain and the transmembrane domain.

In an embodiment of the recombinant vector for use according to the invention, the HA stem polypeptide expressed comprises one or more linkers as described herein; preferably the linker amino acid sequence is GGGG (SEQ ID NO: 2)

In an embodiment of the recombinant vector for use according to the invention, the HA stem polypeptide expressed contains a trimerization domain; preferably the trimerization domain is a GCN4 domain or is a foldon domain; more preferably the trimerization domain is a GCN4 domain; even more preferably the GCN4 domain is placed inside the HA2 ectodomain; yet even more preferably, the GCN4 domain is placed inside the HA2 section in the location of the natural stem-trimer interface as described herein.

For the invention, such placing of the trimerization domain inside the HA2 ectodomain, may be in place of-, or additional to the amino acids of that section, so that it may constitute a replacement or an insertion.

In an embodiment of the recombinant vector for use according to the invention, the Influenza virus HA stem polypeptide expressed has the amino acid sequence selected from one of SEQ ID NO's: 4, 6, 8, 10, and 12.

As the skilled person will recognise, the Influenza virus HA stem polypeptides of SEQ ID NO's: 4, 6, 8, 10, and 12 all have the same general layout, which is further detailed in Tables 1A and 1B. In short:
the 16 aa of the native HA protein signal sequence are replaced by the 25 aa of the signal sequence from CD5;
the HA head domain is missing, and instead the HA1 N- and C-terminal stem domain segments are connected by the aa linker of SEQ ID NO: 2, whereby the HA1 N-terminal segment is placed before (i.e. N-terminal from) the HA1 C-terminal segment;
the Arginine residue at H3 numbering residue 329 was replaced by a Glutamine to prevent HA1-HA2 cleavage;
in the middle of the HA2 ectodomain, the GCN4 trimerization domain was introduced, and the 15 corresponding amino acids of the stem-trimer interface in that site of HA2 were deleted; and
both the transmembrane- and the cytoplasmic domain of HA2 are comprised.

In the HA stem polypeptides of SEQ ID NOs: 4, 6, 8, 10, and 12, the segments of the HA stem domain, the transmembrane domain and the cytoplasmatic domain are specific for H1, H3, H5, H7 and H9, respectively. Also the H1, H3 and H9 stem domain sequences are consensus sequences, determined from aligning a number of recent isolates of Influenza A virus: H1 and H3 from Swine Influenza virus (SIV) isolates, and H9 from Avian Influenza virus (AIV) isolates. The H5 HA stem sequence was taken from AIV strain: H5N1 A/Vietnam/1203/2004 (GenBank: ABW90134); and the H7 HA stem sequence was taken from the AIV strain: H7N9 A/Anhui/1-YK_RG05/2013 (GenBank: AKU41079).

Further, a number of aa mutations were introduced to stabilise the polypeptide, and/or to increase its solubility; these were applied as described in WO 2013/079473.

Over the indications given in Table 1A, there is a small difference in the H7 HA stem polypeptide (SEQ ID NO: 10) as compared to those of SEQ ID NO's: 4, 8, and 12 (respectively the H1, H5, and H9 HA stem polypeptides), in that the HA1 N-terminal stem segment is 1 aa longer, and the HA2 Fragment 2 is 1 aa shorter. The H3 HA stem polypeptide (SEQ ID NO: 6) has the same small difference in the HA2 Fragment 2, but a more significant difference in the HA1 N-terminal stem segment which is 10 aa longer; this is represented in Table 1B.

TABLE 1
A: Detailed common layout of SEQ ID NO's: 4, 8, 10, and 12

| | SEQ ID aa nrs | H3 numbering |
|---|---|---|
| CD5 heterologous signal sequence | 1-24 | — |
| HA1 N-terminal stem segment | 25-59 | 11-45 |
| 4x Glycine linker | 60-63 | — |
| HA1 C-terminal stem segment | 64-86 | 307-329 |
| HA2 ectodomain, 1^{st} part | 87-161 | 330-404 |
| Trimerization domain | 162-176 | — |
| HA2 ectodomain, 2^{nd} part | 177-272 | 420-514 |
| Transmembrane domain | 273-295 | 515-537 |
| Cytoplasmatic domain | 296-308 | 538-550 |

TABLE 1
B: Detailed layout of SEQ ID NO: 6

| | SEQ ID aa nrs | H3 numbering |
|---|---|---|
| CD5 heterologous signal sequence | 1-24 | — |
| HA1 N-terminal stem segment | 25-69 | 11-55 |
| 4x Glycine linker | 60-63 | — |
| HA1 C-terminal stem segment | 64-86 | 317-339 |
| HA2 ectodomain, 1^{st} part | 87-161 | 340-414 |
| Trimerization domain | 162-176 | — |
| HA2 ectodomain, 2^{nd} part | 177-272 | 430-524 |
| Transmembrane domain | 273-295 | 525-547 |
| Cytoplasmatic domain | 296-308 | 548-560 |

In an embodiment of the recombinant vector for use according to the invention, the target is a human; preferably the human target is a young-, old-, sick-, or immunocompromised human.

In an embodiment of the recombinant vector for use according to the invention, the target is an animal; preferably the animal is selected from the group consisting of an avian, porcine, canine, equine, or mustelid. More preferably:
the avian is selected from chicken, turkey, duck, goose, quail, pheasant, partridge, and ostrich;
the porcine is selected from: a wild or a domestic pig, wild boar, babirusa, and warthog;
the canine is a dog;
the equine is a horse;
the mustelid is selected from ferret and mink.

Even more preferably, the target is a swine or a chicken.

In an embodiment of the recombinant vector for use according to the invention, the antibodies against an Influenza virus HA head domain are maternally derived antibodies (MDA's).

As described, the recombinant vector for use according to the invention is capable of expressing the Influenza virus HA stem polypeptide for the invention, because it comprises a nucleic acid sequence that encodes the HA stem polypeptide. In most case the encoding nucleic acid will be heterologous for the vector.

In an embodiment of the recombinant vector for use according to the invention, the nucleic acid that encodes the Influenza virus HA stem polypeptide is codon optimised.

Codon optimisation is well-known, and is applied to improve the expression level of the HA stem polypeptide in a context that differs from that of the origin of the polypeptide. It involves the adaptation of a nucleotide sequence to encode the intended amino acids, but by way of a nucleotide sequence that matches the codon preference (the tRNA repertoire) of the recombinant vector, of the host cell, or of the target organism in which the sequence will be expressed. Consequently, the nucleotide mutations applied are silent.

In an embodiment of the recombinant vector for use according to the invention, the HA stem polypeptide is encoded by a nucleic acid sequence that is codon optimised towards the target organism; preferably that target organism is selected from: human, avian, porcine, canine, equine, and mustelid.

In an embodiment of the recombinant vector for use according to the invention, the HA stem polypeptide is encoded by a nucleic acid sequence selected from one of SEQ ID NO's: 3, 5, 7, 9, and 11.

As the skilled person will realise, the nucleotide sequences indicated in SEQ ID NOs 3, 5, 7, 9, and 11, refer to a DNA nucleic acid, and refer to the ‘coding strand’. For the complementary DNA strand, the ‘template’ strand, the sequence is the inverse-complement.

Also, as is self-evident, when the recombinant vector for use according to the invention comprises an RNA nucleic acid for expressing the HA stem polypeptide, then the same coding sequences apply as in SEQ ID NOs 3, 5, 7, 9, and 11, except that any T is replaced by U.

Further, SEQ ID NOs 3, 5, 7, 9, and 11, or the corresponding sequences as RNA nucleic acid, encode the HA stem polypeptides of SEQ ID NO's: 4, 6, 8, 10, and 12, respectively.

As described, the recombinant vector for use according to the invention can have different forms, ranging from nucleic acid molecules like DNA or RNA, to more complex structures such as virus-like particles and replicon particles, up to replicating recombinant micro-organisms such as a virus.

Therefore in an embodiment, the recombinant vector for use according to the invention is selected from a nucleic acid, a virus, and a replicon particle (RP).

In an embodiment of the recombinant vector for use according to the invention, the vector is a nucleic acid.

In an embodiment of the recombinant vector for use according to the invention wherein the vector is a nucleic acid, the nucleic acid is a eukaryotic expression plasmid.

A eukaryotic expression plasmid, usually of DNA, has the appropriate signals for expression of a heterologous gene that is inserted into the plasmid, under the operational control of a promoter that is active in a eukaryotic cell. The plasmid can then be inserted into a eukaryotic host cell or host organism by some method of transfection, e.g. using a biochemical substance as carrier, by mechanical means, or by electroporation, and will commence the expression of the heterologous gene insert. Typically such expression will be transient, as the plasmid lacks signals for stable integration into the genome of a host cell; consequently such a plasmid will typically not transform or immortalise the host or the host cell. All these materials and procedures are well known in the art and are described in handbooks. Such eukaryotic expression plasmids are commercially available from a variety of suppliers, for example the plasmid series: pcDNA™, pCR3.1™, pCMV™, pFRT™, pVAX1™, pCI™, Nanoplasmid™, pCAGGS etc.

In a preferred embodiment the eukaryotic expression plasmid is a plasmid of the pFRT (ThemoFisher) or pCAGGS plasmids (Niwa et al., 1991, Gene, vol. 108, p. 193-199).

A eukaryotic expression plasmid can comprise several features for regulation of expression, purification, etc. One possible signal is an antibiotic resistance gene, which can be used for selection during the construction and cloning process. However when intended for administration to a human or animal target, such antibiotic selection is not desired for fear of generating antibiotic resistance.

In a preferred embodiment of the recombinant vector for use according to the invention, wherein the vector is a nucleic acid, and the nucleic acid is a eukaryotic expression plasmid, the plasmid does not contain an antibiotic resistance gene.

The recombinant vector for use according to the invention, in the form of a eukaryotic expression plasmid, can be delivered to a host cell or target organisms, where it will express the HA stem polypeptide for the invention in the host cell. Delivery of the expression plasmid can be in several ways, e.g. by mechanical or chemical means, as naked DNA, or encapsulated with an appropriate (nanoparticulate) carrier, such as a protein, polysaccharide, lipid or a polymer. Well-known examples of nucleic acid carriers are dendrimers, lipid nanoparticles, cationic polymers and protamine.

A special form of the recombinant vector for use according to the invention, as a eukaryotic expression plasmid, is when the plasmid provides for the delivery of replicon RNA.

Therefore in an embodiment of the recombinant vector for use according to the invention, wherein the vector is a nucleic acid and the nucleic acid is a eukaryotic expression plasmid, the plasmid encodes a replicon RNA.

A “replicon RNA”, also known as: self-amplifying mRNA, is a self-replicating RNA which contains, in addition to the nucleic acid encoding the HA stem polypeptide for the invention, elements necessary for RNA replication, such as a replicase gene. However, unlike a replicon particle (RP), a replicon RNA is not packaged by viral structural proteins, and is thus less efficient at entering host cells.

The replicon RNA-encoding expression plasmid can be delivered to a host cell in the same way as a protein-expressing plasmid. In this case, no structural viral proteins are co-provided in trans, so that the replicon RNA will not be packaged into an RP.

Vaccination with a eukaryotic expression plasmid encoding replicon RNA provides an advantage over vaccination with a eukaryotic expression plasmid expressing protein, because the replicon RNA provides for an amplification step: the translation of replicase makes the replicon RNA produce subgenomic messenger RNA encoding the HA stem polypeptide. This results in the expression of high amounts of the HA stem polypeptide for the invention in the host cell, respectively in the target.

In a preferred embodiment of the recombinant vector for use according to the invention, wherein the vector is a nucleic acid, the nucleic acid is a eukaryotic expression plasmid, and the plasmid encodes a replicon RNA, the replicon RNA is an Alphavirus-based replicon RNA; more preferably the Alphavirus-based replicon RNA is a Venezuelan equine encephalitis virus (VEEV) based replicon RNA.

An example of a eukaryotic expression plasmid encoding a VEEV replicon RNA is e.g. a pVAX plasmid comprising VEEV non-structural protein genes 1-4, driven by a eukaryotic promoter such as a human CMV immediate early gene 1 promoter.

A specific example of such a plasmid is based on the pVAX plasmid (ThermoFisher), for example ‘pVAX-CMV-T7-HHR-VEEV-dPS-Rep’, as presented in SEQ ID NO: 12. This specific plasmid has 10.709 base pairs, and its composition is as described in Table 2.

TABLE 2
Composition of plasmid pVAX-CMV-T7-HHR-VEEV-
dPS-Rep as represented in SEQ ID NO: 12

| | SEQ ID NO: 12, |
|---|---|
| | nucleotide numbers |
| hCMV IE1 promoter | 33-620 |
| T7 polymerase promoter | 634-651 |
| Hammerhead ribozyme | 651-709 |
| VEEV 5′ UTR | 710-753 |
| VEEV nsP1 | 754-2358 |
| VEEV nsP2 | 2359-4740 |
| VEEV nsP3 | 4741-6411 |
| VEEV nsP4 | 6412-8232 |
| Insertion site for gene to be expressed | 8296-8297 |
| VEEV 3′ UTR | 8308-8476 |
| Bovine growth hormone polyA signal | 8535-8759 |
| Kanamycin resistance gene | 8932-9726 |
| pUC origin | 10026-10699 |

In an alternate embodiment of the recombinant vector for use according to the invention, wherein the vector is a nucleic acid, the nucleic acid is an RNA molecule.

The RNA molecule for the invention can have different forms and functions, for example can be an mRNA or can be a replicon RNA.

A recombinant vector for use according to the invention, as an RNA molecule can be delivered to the target or to a host cell in different ways, e.g. by mechanical or chemical means, or encapsulated with an appropriate (nanoparticulate) carrier, such as a protein, polysaccharide, lipid or a polymer, as described. To stabilise the RNA certain chemical modifications may be applied, e.g. to the nucleotides or to their backbone, or the incorporation of nucleotide-analogues.

In an embodiment of the recombinant vector for use according to the invention wherein the vector is a nucleic acid and the nucleic acid is an RNA molecule, the RNA molecule is an mRNA.

An “mRNA” is well-known in the art, and typically has a 5′ 7mG cap and a 3′ poly-A tail. An mRNA can be delivered to a eukaryotic host organism or host cell by way of transfection and/or by using an appropriate carrier, e.g. a polymer or a cationic lipid.

In an embodiment of the recombinant vector for use according to the invention wherein the vector is a nucleic acid and the nucleic acid is an RNA molecule, the RNA molecule is a replicon RNA.

The replicon RNA can be produced in vitro e.g. using the pVAX-CMV-T7-HHR-VEEV-dPS-Rep plasmid as described above, and then administered to a host cell or a target organism, using any suitable method.

Recombinant vectors for the expression and delivery of a heterologous antigen in the form of replicating recombinant virus vectors, are well-known in the art. These provide for an efficient method of vaccination, as the viral vector replicates and amplifies in the target. Assembly and modification of a recombinant vector virus is routine using standard molecular biological techniques.

Many different virus species have been used over time as recombinant vector, and for a variety of human and animal targets.

Therefore in an embodiment of the recombinant vector for use according to the invention, the recombinant vector is a virus.

In an embodiment of the recombinant vector for use according to the invention wherein the vector is a virus, the virus is selected from a Herpesvirus, a Poxvirus, a Retrovirus, a Paramyxovirus, a Rhabdovirus and an Adenovirus.

For use in a human target, the recombinant vector virus is preferably an Adenovirus, a Rhabdovirus, e.g. Vesicular stomatitis virus, or a Paramyxovirus, e.g. Measles virus. For use in an avian target the recombinant vector virus is preferably a Herpesvirus, more preferably a Herpesvirus of Turkeys (HVT) or a Marek's disease virus of serotype 1 or 2. For use in a porcine target, the recombinant vector virus is preferably a herpesvirus, e.g. Pseudorabies virus (Suid herpesvirus 1).

Because a recombinant vector virus is relatively large, not only single- but even multiple inserts of heterologous genes for expression are possible. Examples of recombinant viral vectors expressing and delivering an Influenza HA gene are described: for HVT as vector in WO 2012/052384 and EP19218804.3. For Newcastle disease virus (NDV; an avian paramyxovirus) as vector, an example is described in: WO 2007/106882.

For the construction of a recombinant viral vector, typically an expression cassette is inserted into a locus in the vector's genome. Different techniques are available to control the locus and the orientation of that insertion. For example by using the appropriate flanking sections from the genome of the vector to direct the integration of the cassette by a homologous recombination process, e.g. by using overlapping cosmids as described in U.S. Pat. No. 5,961,982. Alternatively the integration may be done by using the CRISPR/Cas technology.

An ‘expression cassette’ is a nucleic acid fragment comprising at least one heterologous gene and a promoter to drive the transcription of that gene, to enable the expression of the encoded protein. The termination of the transcription may be provided by sequences provided by the genomic insertion site of the cassette, or the expression cassette can itself comprise a termination signal, such as a transcription terminator. In such a cassette, both the promoter and the terminator need to be in close proximity to the gene of which they regulate the expression; this is termed being ‘operatively linked’, whereby no significant other sequences are present between them that would intervene with an effective start-, respectively termination of the transcription. As will be apparent to a skilled person, an expression cassette is a self-contained expression module, therefore its orientation in a vector virus genome is generally not critical.

The recombinant vector for use according to the invention can also be delivered and expressed to a target by way of a macro-molecular structure that resembles a virion. Examples are virus-like particle (VLPs) or replicon particles (RPs). Known as ‘single cycle’ infectious particles, these contain features necessary to infect a host cell, and express the heterologous gene it carries, encoding the polypeptide, however, as a built-in safety feature, they will typically not be capable of full viral replication, for lack of (relevant parts of) the viral genome from which they were constructed.

“RPs” are well-known, and several RPs have been developed as a platform for the expression and delivery of a variety of proteins. Favourable basis for an RP is an Alphavirus, because of its broad host-range and rapid replication. Of course appropriate safety measures need to be taken to attenuate and control the infection of such RPs, as some Alphaviruses are highly pathogenic in their wildtype form. For a review, see: Kamrud et al., 2010, J. Gen. Virol., vol. 91, p. 1723-1727, and: Vander Veen, et al., 2012, Anim. Health Res. Rev., vol. 13, p. 1-9.

Therefore in an embodiment of the recombinant vector for use according to the invention, the vector is an RP. Preferably the RP is an Alphavirus RP; more preferably the Alphavirus RP is a VEEV RP.

Preferred Alphavirus RPs are based on VEEV, which have been applied as recombinant vector vaccine for human, swine, poultry, and fish. Methods and tools to construct, test, and use VEEV-based Alphavirus RPs are well-known and available, see for example: Pushko et al., 1997, Virology, vol. 239, p. 389-401, and: WO 2019/110481. Preferred VEEV RP technology is the SirraVax℠ RNA Particle technology (Harrisvaccine).

In an embodiment the pVAX-CMV-T7-HHR-VEEV-dPS-Rep plasmid is used to produce RPs: RNA is produced from the plasmid which is then transfected into the host cells together with helper RNAs encoding in trans the VEEV structural proteins.

In an embodiment of the recombinant vector for use according to the invention, one or more of the conditions apply, selected from the group consisting of:
the disease caused by influenza virus, is caused by an Influenza A virus or by an Influenza B virus; preferably the disease is caused by an Influenza A virus;
the HA stem polypeptide expressed is derived from an Influenza A virus or from an Influenza B virus; preferably the HA stem polypeptide is derived from an Influenza A virus HA protein selected from any one of the serotypes: H1 through H18; more preferably the HA stem polypeptide is derived from an Influenza A virus HA protein selected from any one of the serotypes: H1, H3, H5, H7, and H9;
the HA stem polypeptide expressed is a consensus sequence;
the general order of the constituting parts of the HA stem polypeptide expressed is, from N-terminal to C-terminal:
   the HA1 N-terminal stem segment,
   the HA1 C-terminal stem segment,
   the HA2 ectodomain,
   the transmembrane domain, and
   the cytoplasmic domain;
the HA stem polypeptide expressed contains a linker sequence in between one or more of:
   the HA1 N-terminal stem segment and the HA1 C-terminal stem segment;
   the HA1 C-terminal stem segment and the HA2 ectodomain; and
   the HA2 ectodomain and the transmembrane domain;
the HA stem polypeptide expressed comprises one or more linkers according to the invention; preferably the linker amino acid sequence is GGGG (SEQ ID NO: 2);
the HA stem polypeptide expressed contains a trimerization domain; preferably the trimerization domain is a GCN4 domain or is a foldon domain; more preferably the trimerization domain is a GCN4 domain; even more preferably the GCN4 domain is placed inside the HA2 ectodomain; yet even more preferably, the GCN4 domain is placed inside the HA2 section in the location of the natural stem-trimer interface as described herein;
the HA stem polypeptide expressed has the amino acid sequence as presented in one of SEQ ID NO's: 4, 6, 8, 10, and 12;
the target is a human; preferably the human target is a young-, old-, sick-, or immunocompromised human;
the target is an animal; preferably the animal is selected from the group consisting of avian, porcine, canine, equine, or mustelid; more preferably:
   the avian is selected from chicken, turkey, duck, goose, quail, pheasant, partridge, and ostrich;
   the porcine is selected from: a wild or a domestic pig, wild boar, babirusa, and warthog;
   the canine is a dog;
   the equine is a horse;
   the mustelid is selected from ferret and mink;
the target is a swine or a chicken;
the antibodies against an Influenza virus HA head domain are maternally derived antibodies (MDA's);
the nucleic acid that encodes the HA stem polypeptide is codon optimised;
the HA stem polypeptide is encoded by a nucleic acid sequence that is codon optimised towards the target organism; preferably that target organism is selected from: human, avian, porcine, canine, equine, and mustelid;
the HA stem polypeptide is encoded by a nucleic acid sequence as presented in SEQ ID NO's: 3, 7, 9, and 11; and
the recombinant vector for use according to the invention is selected from a nucleic acid, a virus, and an RP; preferably:
   the nucleic acid is a eukaryotic expression plasmid or an RNA molecule;
   the virus is selected from a Herpesvirus, a Poxvirus, a Retrovirus, a Paramyxovirus, a Rhabdovirus and an Adenovirus; or
   the RP is an Alphavirus RP.

In an embodiment of the recombinant vector for use according to the invention, the HA stem polypeptide expressed has the amino acid sequence as presented in one of SEQ ID NO's: 4, 6, 8, 10, and 12; the target is a swine or a chicken; the antibodies against an Influenza virus HA head domain are maternally derived antibodies; the nucleic acid that encodes the HA stem polypeptide is codon optimised towards the target organism; and the vector is selected from a nucleic acid, a virus, and an RP.

The recombinant vector for use according to the invention can advantageously be used to deliver and express the Influenza virus HA stem polypeptide for the invention to a target, e.g. as a way to vaccinate that target. This involves at some stage the introduction of that vector into a suitable host cell. Depending on the type of vector applied, that introduction into a host cell may require a carrier, some method of transfection, or may be guided by the vector itself, as described above. Nevertheless, once the vector is inside the host cell, the HA stem polypeptide is expressed, and thereby the host cell infected or transfected with the recombinant vector, itself can be used for the invention, e.g. as the infected or transfected host cell may be used for the vaccination of a target.

A “host cell” for the invention, is a cell that allows the expression of the HA stem polypeptide for the invention, from the recombinant vector for use according to the invention, after the introduction of that vector into said host cell, e.g. by way of transfection or infection.

A host cell for the invention can be a primary cell, for example a cell in a target organisms, or a cell that is kept in vitro, either as cells in a suspension, in a monolayer, or in a tissue. Typically primary cells can only perform a small and limited number of cell-divisions when in vitro.

Alternatively the host cell can be an immortalised cell, for example as from an established cell-line, which can grow and divide almost indefinitely. Depending on the type of the host cell, the expression of the HA stem polypeptide for the invention will include more or less extensive post-translational processing, such as e.g. signal peptide cleavage, disulphide bond formation, glycosylation, and/or lipid modification.

The primary- or the immortalised host cell can be of the same- or from a different species as the target for the recombinant vector for use according to the invention.

Much used host cells are fibroblasts and lymphocytes. In case of the use of HVT as recombinant virus vector for use in the invention, the host cells are preferably primary chicken embryo fibroblasts (CEF's), which can be used and stored as described, see e.g. WO 2019/121888.

The host cell for the invention is preferably an immortalised avian cell. Several immortalised avian cell-lines have been described, for example in WO 97/044443 and WO 98/006824; more preferably the immortalised avian host cell for the invention is an immortalised CEF; even more preferably an immortalised CEF as disclosed in WO 2016/087560.

As described, the recombinant vector for use according to the invention and the host cell for the invention, can advantageously be used in a vaccine to reduce infection or disease caused by Influenza virus.

Therefore, in a further aspect the invention relates to a vaccine for use in reducing infection or disease caused by influenza virus in a target that has antibodies against an Influenza virus HA head domain at the time of vaccination, the vaccine comprising the recombinant vector for use according to the invention, or a host cell comprising said recombinant vector, and a pharmaceutically acceptable carrier.

A “vaccine” is well-known to be a composition comprising an immunologically active compound, in a pharmaceutically acceptable carrier. The ‘immunologically active compound’, or ‘antigen’ is a molecule that is recognised by the immune system of the inoculated target and induces a protective immunological response from the humoral- and/or the cellular immune system of the target.

The vaccine for use according to the invention is a vaccine ‘against Influenza’ and provides for the reduction of signs of infection or of disease, as caused by an influenza virus, as described herein above.

Specifically, the vaccine for use according to the invention is effective in a target with pre-existing antibodies against an Influenza virus HA head domain, as described herein above.

A “pharmaceutically acceptable carrier” is well-known to aid in the stabilisation and the administration of a vaccine, while being harmless and well-tolerated by the target. Such a carrier can for instance be sterile water or a sterile physiological salt solution. In a more complex form the carrier can e.g. be a buffer, which can comprise further additives, such as stabilisers or conservatives. Details and examples are for instance described in well-known handbooks such as: “Remington: the science and practice of pharmacy” (2000, Lippincott, USA, ISBN: 683306472), and: “Veterinary vaccinology” (P. Pastoret et al. ed., 1997, Elsevier, Amsterdam, ISBN 0444819681).

For the present invention, when the vaccine comprises a cell-associated HVT recombinant viral vector, then the pharmaceutically acceptable carrier is preferably a mixture of culture medium comprising serum and DMSO. This carrier also provides for the stabilisation of the HVT vector-infected host cells during freezing and frozen storage. The serum can e.g. be foetal- or new-born calf serum.

Similarly, when the vaccine for use according to the invention comprises a nucleic acid or an RP, the pharmaceutically acceptable carrier can be a simple buffer, e.g. phosphate buffer with 5% w/v sucrose.

Further an additional carrier can be added to stabilise and/or deliver the recombinant vector for use according to the invention, e.g. to encapsulate with an appropriate (nanoparticulate) carrier, such as a protein, polysaccharide, lipid or a polymer, as described. Preferably the additional carrier for an RP for the invention comprises a nanogel that is a biodegradable polyacrylic polymer as described in WO 2012/165953.

The vaccine for use according to the invention can comprise additional immunoactive components. This can serve to enhance the immune protection already provided, or to expand it to other pathogens.

Therefore, in an embodiment, the vaccine for use according to the invention comprises at least one additional immunoactive component.

Such an “additional immunoactive component” may be an antigen, an immune enhancing substance, a cytokine, a further vaccine, or any combination thereof. This provides advantages in terms of cost, efficiency and welfare. Alternatively, the vaccine for use according to the invention, may itself be added to a vaccine.

A further advantageous effect of the reduction of Influenza viral load in a target, by the vaccine for use according to the invention, is the prevention or reduction of virus shedding by infected targets, and thereby of the spread of Influenza virus, both vertically to offspring, and horizontally within a flock or population and within a geographical area. Consequently, the use of a vaccine for use according to the invention leads to a reduction of the prevalence of Influenza virus.

Therefore further aspects of the invention are:
the use of a vaccine for use according to the invention for reducing the prevalence of Influenza virus in a population or in a geographical area; and
the vaccine for use according to the invention for reducing the prevalence of Influenza virus in a population or in a geographical area.

The vaccine for use according to the invention is prepared by well-known methods, for example as described and exemplified herein, e.g. comprising the step of admixing of the recombinant vector for use according to the invention, or of the host cell for the invention, with the pharmaceutically acceptable carrier.

In addition various other compounds can be added to a vaccine for use according to the invention, e.g. stabilisers, carriers, adjuvants, diluents, emulsions, etc. Such additives are described in well-known handbooks such as: “Remington”, and “Veterinary Vaccinology” (both supra).

This way the efficacy of a vaccine for use according to the invention can be further optimised when needed by a skilled person, using routine techniques.

General techniques and considerations that apply to the manufacture of vaccines under well-known standards for pharmaceutical production are described for instance in governmental directives and regulations (Pharmacopoeia, 9CFR) and in well-known handbooks (“Veterinary vaccinology” and: “Remington”, both supra). Commonly such vaccines are prepared sterile, and are prepared using excipients of pharmaceutical quality grade.

Such preparations will incorporate microbiological tests for sterility, and absence of extraneous agents, and may include studies in vivo or in vitro for confirming efficacy and safety. After completion of the testing for quality, quantity, sterility, safety and efficacy, the vaccine can be released for sale. All these are well-known to a skilled person.

Depending on the route of application of the vaccine for use according to the invention, it may be necessary to adapt the vaccine's composition. This is well within the capabilities of a skilled person, and generally involves the fine-tuning of the efficacy or the safety of the vaccine. This can be done by adapting the vaccine dose, quantity, frequency, route, by using the vaccine in another form or formulation, or by adapting the other constituents of the vaccine (e.g. a stabiliser or an adjuvant).

Preferably a vaccine for use according to the invention is formulated as an injectable liquid, suitable for injection either in ovo, intradermal, or parenteral. The injectable liquid can e.g. be a suspension, solution, dispersion, or emulsion.

In an embodiment, the vaccine for use according to the invention is for administration by parenteral route. Preferably by intramuscular- or subcutaneous route.

In an embodiment the vaccine for use according to the invention is for administration by intradermal route. More preferably the intradermal route of administration is applied to a swine target.

In a further aspect the invention relates to the use of the recombinant vector for use according to the invention, or of a host cell comprising said recombinant vector, and/or of the vaccine for use according to the invention, for reducing infection or disease caused by influenza virus in a target that has antibodies against an Influenza virus HA head domain at the time of vaccination.

Similarly, In a further aspect the invention relates to a method for reducing infection or disease caused by influenza virus in a target that has antibodies against an Influenza virus HA head domain at the time of vaccination, the method comprising administering to said target the recombinant vector for use according to the invention, a host cell comprising said recombinant vector, and/or the vaccine for use according to the invention.

In a preferred embodiment of the use according to the invention, or of the method for reducing infection according to the invention, the vaccine for use according to the invention comprises in addition a vaccine comprising a full length HA protein.

In this combination, the vaccine for use according to the invention can provide an early immunity against Influenza in the context of MDA, and in addition can provide Influenza virus immunity of long duration.

The volume per target dose of the vaccine for use according to the invention can be optimised according to the intended route of application: in ovo inoculation is commonly applied with a dose of between about 0.01 and about 0.5 ml/egg, and parenteral injection is commonly done with a dose of between about 0.1 and about 10 ml/target.

Determination of what is an immunologically effective amount of the vaccine for use according to the invention, or the optimisation of the vaccine's volume per dose, are both well within the capabilities of the skilled artisan.

The dosing regimen for applying the vaccine for use according to the invention to a target organism can be in single or multiple doses, in a manner compatible with the formulation of the vaccine, and in such an amount as will be immunologically effective.

Preferably, the regimen for administering a vaccine for use according to the invention is integrated into existing vaccination schedules of other vaccines that the target may require, in order to reduce stress to the target and to reduce labour costs. These other vaccines can be administered in a simultaneous, concurrent or sequential fashion, in a manner compatible with their licensed use.

Preferably the vaccine for use according to the invention is only administered once, as a single-shot.

When the target for treatment with the recombinant vector for use according to the invention, with the host cell comprising said vector, or with the vaccine for use according to the invention, is an avian, the treatment is preferably applied at very early age: at the day of hatch (“day 1”), or in ovo, e.g. at 18 days of embryonic development, all well-known in the art.

The invention will now be further described with reference to the following, non-limiting, examples.

### EXAMPLES

### Example 1: Preparation of Recombinant Constructs

### 1.1. HVT Vaccines:

HVT viral vector vaccines were prepared using methods for transfection, recombination, selection and amplification essentially as described in WO 2012/052384 and WO 2016/102647. In HVT, the full H5 HA gene and the H5 HA stem encoding construct (SEQ ID NO: 7) were driven by the PRV gB gene promoter, and the expression cassette was inserted into the Us2 locus of the HVT genome.

### 1.2. Replicon Particles

VEEV RP's were constructed, produced and selected, using the split-helper system, as described in WO 2005/113782, WO 2008/156829, and: Kamrud et al. (2010, J. Gen. Virol., vol. 91, p. 1723-1727). The inserts used for the RP's were the H5 HA stem encoding construct (SEQ ID NO: 7) and the H9 HA stem encoding construct (SEQ ID NO: 11) for experiments in chickens, and the H1 HA stem encoding construct (SEQ ID NO: 3) for use in swine. Details of the use in swine-animal experiments were as described in WO 2019/110481.

### 1.3. Plasmids

VEEV Replicon RNA samples had essentially the same HA stem encoding insert as used in the HVT and the RP experiments, except that these were delivered using expression plasmids of the pFRT or pVAX1 series as vector.

Transformed E. coli K12 containing the pFRT or pVAX plasmids were amplified in LB medium. Plasmid DNA isolation was conducted using EndoFree™ Plasmid Kits (QIAGEN). Plasmid DNA was eluted in water-for injection or TE buffer.

### Example 2: Vaccination-Challenge Experiment in SPF and MDA+ Chickens

### 2.1. INTRODUCTION

### 2.1.1. Objective

The objective was to evaluate different types of Influenza vaccines for their capacity to provide protection in 1-day-old MDA+ or SPF chickens against experimental challenge infection with a highly pathogenic avian influenza (HPAI) H5N1 virus. The challenge was performed at 2 and 3 weeks p.v. for the SPF chicks, and at 4 or 5 weeks p.v. (p.v.) for the MDA+ chicks, to determine the various aspects of vaccine-efficacy in targets with- or without pre-existing antibodies and of different titres.

As a model to test Influenza vaccine efficacy, the most reliable parameters are the scoring of target animal mortality and challenge virus replication and -excretion. This is e.g. described in the OIE Manual of Diagnostic Tests and Vaccines for Terrestrial Animals 2015, chapter 2.3.4. Avian Influenza.

### 2.1.2. Study Design

For this study, n=80 (+n=4 spare) one-day-old healthy MDA+ and n=55 (+n=4 spare) one-day-old healthy SPF layer chickens were used and transported to a contract research organisation (CRO) at day 0, D8, D14 and D21 of the study. SPF chicks were negative for (among many others) antibodies against Influenza virus.

For this study, n=20 one-day-old healthy MDA+and n=10 one-day-old healthy SPF layer chickens were used for day 0, 7, 14 and 21 blood sampling, which were transported to the CRO at D0, D8, D14 and D21 of the study.

Chicks were distributed over 10 groups and immunized at the day of arrival as indicated in Table 3 During the study, chickens were housed in two animal rooms and kept in separate pens per treatment group (T01-T05, T06-T10).

On the day of challenge, chickens were sampled for serology (study day 35). All chickens were inoculated with challenge virus HPAI H5N1 A/turkey/Turkey/01/2005 (clade 2.2.1). For 10 days post challenge infection, chickens were monitored for clinical disease and mortality at least twice daily, and choanae swabs were collected once daily on days 1, 2, 3, 5, and 7 post challenge infection (dpci). On dpci 10, the study was ended by euthanizing all chickens that survived the challenge infection. The study ended at D45.

TABLE 3
Group assignment and animal handlings

| | | | | Arrival | Immunization | | | |
|---|---|---|---|---|---|---|---|---|
| | | | Chickens | at CRO | | | | Dose |
| Group | Room | Status | (n) | (Day) | Day | Vaccine | Route | (mL) |
| T01 | A | MDA+ | 10 | 0 | 0 | None | — | na |
| T02 | | MDA+ | 15 | 0 | 0 | HVT + full H5 HA | SC | 0.2 |
| T03 | | MDA+ | 15 | 0 | 0 | HVT-H5 HA stem | SC | 0.2 |
| T04 | | MDA+ | 10 | 8 | 8 | None | — | — |
| T05 | | MDA+ | 15 | 8 | 8 | HVT + full H5 HA | SC | 0.2 |
| T06 | | MDA+ | 15 | 8 | 8 | HVT-H5 HA stem | SC | 0.2 |
| T07 | B | SPF | 15 | 14 | 14 | HVT + full H5 HA | SC | 0.2 |
| T08 | | SPF | 15 | 14 | 14 | HVT-H5 HA stem | SC | 0.2 |
| T09 | | SPF | 10 | 21 | 21 | None | — | — |
| T10 | | SPF | 15 | 21 | 21 | HVT + full H5 HA | SC | 0.2 |

| | | | | |
|---|---|---|---|---|
| T = 0 blood | MDA+ | 10 | 0 | T = 0, 7, 14 and 21 bleeds were performed inhouse and |
| T = 7 blood | MDA+ | 10 | 7 | then transported together with the chickens to the CRO |
| T = 14 blood | SPF | 5 | 14 | |
| T = 21 blood | SPF | 5 | 21 | |

### 2.2. MATERIALS AND METHODS

### 2.2.1. Test Articles

HVT vaccines were used as a suspension of infected primary chicken embryo fibroblasts (CEFs). Materials were stored in liquid nitrogen until use, and were then diluted in commercial HVT dilution buffer Solvent CA™ (MSD Animal Health), then kept at ambient temperature, and was used within one hour of reconstitution.

HVT vaccines were used at 2,000 PFU per animal dose of 0.2 ml, administered subcutaneously (sc) in the neck, On study day 0, 8, 14, or 21. One syringe and needle was used per treatment group.

### Test Article: Vector Vaccine of HVT-Full H5 HA Gene Insert

The HVT vector construct used contained the Pseudorabies virus gB gene promoter driving the full H5 HA gene, and a human CMV immediate early gene 1 promoter, driving the NDV F gene, whereby the PRV gB prom. +HA gene insert is as described in WO 2012/052384, and the hCMV IE1 prom. +F gene insert is as described in WO 2016/102647. These respective promoter+gene inserts are inserted tail-head in the Us2 gene locus of the HVT genome. The dual cassette includes a downstream transcription terminator from the hCMV-IE1 gene.

### Test Article: Vector Vaccine of HVT-H5 HA Stem

The HVT vector used to deliver and express the H5 HA stem polypeptide was essentially a construct as described in WO 2012/052384, and contained the Pseudorabies virus gB gene promoter driving the H5 HA stem polypeptide, encoded by the nucleotide sequence of SEQ ID NO: 7, and inserted into the Us2 gene locus of the HVT genome, and was followed by a transcription terminator.

### Challenge Virus: HPAI H5N1 Strain A/turkey/Turkey/1/2005

The challenge virus was kept at −80° C. until dilution to about 6 Log 10 EID50 (i.e. 5 Log 10 TCID50 equivalent) in 0.2 ml. Inoculum was kept on ice until use, and remaining inoculum was back-titrated to determine the actual challenge dose administered.

The challenge virus was administered as 0.1 ml intranasally (one nostril), and 0.1 ml intratracheally.

NB: Work with virulent influenza virus requires appropriate permissions and biosafety precautions.

### 2.2.2. Test Animals

135 White leghorn layer chickens were used in the study, one day old, and of mixed sex, whereby only healthy animals were transported to the CRO. Chickens were excluded (on study day 0) from this study if they appeared not in good health upon arrival. Prior to vaccination, chickens were identified with swifttack labels in the neck as they came to hand, numbered as indicated in Table 3. MDA+: n=80 in study; n=20 for T=0 and T=7 blood and n=4 spare; SPF: n=55 in study, n=10 for T=14 and T=21 blood and n=4 spare.

The MDA+ chicks were the offspring of SPF layer chickens that had been vaccinated with an inactivated and adjuvated AIV vaccine, prepared from HPAI H5N1 A/turkey/Turkey/01/2005 (clade 2.2.1). Consequently, the MDA were homologous to the encoded H5 HA stem polypeptide, to create a worst-case scenario.

One-day-old chickens were immunized on the day of arrival. Non-vaccinated chicken from groups T01, T05 and T08 had an acclimatization period of 14, 28 and 35 days, respectively.

Chickens were group-housed under appropriate conditions, per treatment group (max. n=15) in separate pens with closed walls to prevent physical contact.

### 2.2.3. Experimental Procedures

General health was observed and documented by a bio-technician at least once daily, and a veterinarian was called-in when needed.

Clinical observations for signs of influenza were performed and recorded daily by an animal-technician from the day of challenge virus inoculation until 10 days post inoculation. With the first observation performed prior to inoculation. Clinical observations were performed according to a scoring system with scores of 0-3 for increasing severity for signs of: depression, nasal discharge, sneezing, breathing, skin aberrations, oedema, neurological signs, and diarrhoea.

Blood samples for serology (approximately 2 mL per animal) were collected from the wing vein on study day 20 from all chickens. Serum was isolated after clotting and centrifugation (10 min at 1300 xg). Serum samples were stored at −20° C.

Choanae swab samples were collected on day 36, 37, 38, 40, and 42 from all chickens. Samples were collected using cotton swabs (dry rayon tip, Copan 155C). Directly following sampling, the swabs were agitated in approximately 2 mL of tryptose phosphate buffer supplemented with antibiotics and kept on melting ice during transport to the lab. Chickens found dead were not sampled, but moribund chickens were sampled before being euthanized. In the laboratory, the swabs were squeezed and removed, samples were centrifuged (10 min. 1300 xg) and the supernatant was stored at −80° C.

When a chicken displayed severe symptoms (score 3) of depression, respiratory distress or a neurological disorder on the first observation or moderate symptoms (score 2) of depression, respiratory distress or a neurological disorder on the second observation, it was euthanized based on the criteria on the recognition, assessment, and use of clinical signs as humane endpoints for experimental animals.

After completion of the in-life phase, clinical observations for signs of influenza were summarized per animal and day of study. Per treatment group, median clinical scores were calculated and displayed graphically over time. In addition, the frequency (number of days), sum and distribution of the severity scores were calculated.

A hemagglutination inhibition (HI) assay was performed on serum samples using HA antigen that was homologous to the vaccine- and challenge antigen. Also, samples were subjected to a specific AIV-H5 HA inhibition ELISA (ID Screen® Influenza H5 Antibody Competition (IDVet)). HI titres and ELISA scores from serum samples were summarized per animal and day of study. Per treatment group, mean titres were calculated.

Choanae swab samples were used to perform an influenza real-time qPCR. Ct values were summarized by animal and day of study. Per treatment group, mean Ct values were calculated and displayed graphically over time. In addition, mean peak Ct values and number of days with positive PCR result were calculated.

Tests were found to be valid when: serum samples taken from one day old SPF hatchlings, and from the unvaccinated SPF hatchlings, contained no antibodies directed against AIV-H5. Also, the unvaccinated challenged chickens needed to have died within 10 days after challenge.

### 2.3. RESULTS

A few chicks died during the experiment but prior to challenge. Causes could not be established or were unrelated to the trial.

### 2.3.1. Challenge

To determine the challenge dose, the non-diluted challenge virus, diluted virus prior to challenge and diluted challenge virus that came back from the animal facility after the challenge were subjected to a TCID50 assay. The average of the titres after dilution (prior and post administration) were used to calculate the inoculation dose, which was determined at: 10\hat{ }3.95 TCID50 per animal.

After challenge, mortality and morbidity was monitored for 10 days. All the non-immunized SPF chickens died or had to be euthanized prior to or at day two post challenge, thus, the challenge of HPAI H5N1 was severe and very effective. Challenge of non-immunized AIV MDA+ animals at 4 weeks post hatch resulted in a mortality rate of 70%, which shows that the residual MDA titres still resulted in partial protection of the challenged animals. In contrast, at 5 weeks post hatch the MDA titres were no longer protective.

### 2.3.2. Effect of Vaccination on Survival

In the SPF animals, The HVT-Full H5 HA vaccination resulted in 100% survival in challenged animals at 2 weeks and 3 weeks p.v. Thus, the onset-of-immunity (OOI) of the HVT-Full H5 HA vaccine in SPF animals is <2 weeks. The efficacy of the HVT-H5 HA stem vaccine in SPF animals was only determined at 3 weeks p.v., since its onset was slightly later. The HVT-H5 HA stem resulted in a partial protection of 60%, thus its onset-of-immunity in SPF animals is >3 weeks.

However, in contrast to the results in SPF animals, in AIV MDA+ animals the HVT-Full H5 HA resulted only in 43% and 46% protection at 4 and 5 weeks, respectively. Surprisingly, the HVT-H5 HA stem vaccine resulted in 86% and 93% protection in AIV MDA+animals.

### 2.3.3. Challenge Virus Replication

At days 1, 2, 3, 5 and 7 post challenge choana swabs were taken from non-immunized chickens (n=5) and immunized chickens (n=10), and used to determine challenge virus replication by measuring AIV RNA load in the trachea. The RT-qPCR results were indicated as PCR equivalent virus titres (EID50 equivalents) including standard deviations (SD). The non-immunized SPF chickens had high loads of viral replication in the trachea at day 1 post challenge (on average 10\hat{ }4.3 EID50 equivalents). In non-immunized AIV MDA+ chickens the titres were 10\hat{ }3.2 and 10\hat{ }3.8 EID50 equivalents at 4 and 5 weeks post hatch, respectively.

Titres of challenge virus in SPF animals vaccinated with HVT-Full H5 HA were reduced by 2 Log 10 at 2 weeks p.v. and by >4 Log 10 at 3 weeks p.v. Vaccination of SPF animals with HVT-H5 HA stem vaccine resulted only in a reduction of challenge virus replication by 1 Log 10.

Although vaccination of AIV MDA+ animals had a strong effect on survival, especially for the HVT-H5 HA stem vaccine, there was only a minor effect on challenge virus replication in the trachea. Vaccination of AIV MDA+ animals with HVT-Full H5 HA also had only a minor effect with a reduction in viral load less than 1 Log 10. The HVT-H5 HA stem vaccine resulted in a reduction of challenge virus replication between 1-2 Log 10, both at 4 and 5 weeks p.v.

### 2.3.4. Serological Results

### 2.3.4.1. Hemagglutination Inhibition (HI) Titres

Serum from blood isolated before challenge, was used for an HI assay using an HA antigen. HI titres were determined in duplicate. As expected, the HVT-H5 HA stem vaccine did not induce antibodies that resulted in hemagglutination inhibition, since only antibodies directed to the head domain can inhibit hemagglutination.

All SPF hatch mates (controls, T=0) were negative, same as the non-immunized SPF animals at 2 weeks p.v. HVT-Full H5 HA vaccination resulted in seroconversion of 12 out of 14 animals at 2 weeks p.v. with an average HI titre of 10.2. At three weeks p.v., HVT-Full H5 HA resulted in seroconversion of 13 out of 14 animals with an average HI titre of 52.8.

All AIV MDA+ hatch mates (control, T=0) were positive in the HI test with an average titre of 46.9. Titres dropped to values <2 at 4 weeks and 5 weeks post hatch (controls). Surprisingly, the HVT-Full H5 HA failed to induce any seroconversion at 4 and 5 weeks p.v. in MDA+ chicks upon vaccination.

### 2.3.4.2. AIV-H5 Specific ELISA

A commercial inhibition ELISA test kit (AIV-H5 ELISA, IDVet) was used according to the manufacturer's instructions, to test the serum samples obtained from chicks during the experiment.

Serum from SPF hatch mates taken prior to vaccination showed an inhibition titre of 0%. Also non-immunized SPF animals (negative controls) showed an inhibition of 7%, which is at background level.

ELISA titres in SPF animals following vaccination with the HVT-Full H5 HA vaccine slowly increased from 2-3 weeks p.v.: from inhibitory titres of 35% at 2 weeks, to 57% at 3 weeks p.v.

In contrast to these relatively high antibody titres induced in SPF animals, the vaccination of MDA+ animals with the HVT-Full H5 HA vaccine resulted in only 32% inhibition at 4 weeks and 22% inhibition at 5 weeks p.v. These H5 antibody titres were comparable to the inhibitory anti-H5 titres as observed in the non-immunized MDA+ animals. Consequently, the HVT-Full HA H5 vaccine was severely inhibited by the MDA titres present at the time of vaccination.

The vaccination of SPF animals with the HVT-H5 HA stem vaccine resulted in inhibitory titres of 33% at 3 weeks p.v. So, the HVT-H5 HA stem vaccine induces relatively lower titres in the SPF animals, as compared to the Full HA vector vaccine in SPF a chicks.

Surprisingly however, MDA+ animals vaccinated with the HVT-H5 HA stem vaccine, had inhibitory Elisa titres of 41% at 4 weeks, and of 44% at 5 weeks p.v. These titres were thus higher compared to HVT-H5 HA stem vaccinated SPF animals at 3 weeks p.v. and much higher compared to the non-vaccinated MDA+ controls. Thus, the HVT-H5 HA stem vaccine is not affected by the AIV-H5 MDA titres present at the time of vaccination.

### 2.4. CONCLUSIONS

HVT-Full H5 HA and HVT-H5 HA stem vaccines were evaluated in one-day-old SPF and AIV MDA+ chickens. SPF animals were subjected to a homologous H5N1 challenge at 2 and 3 weeks p.v. and AIV MDA+ animals were challenged at 4 and 5 weeks p.v. Serological responses, mortality and challenge virus replication in the trachea were assessed.

In SPF animals, HVT-Full H5 HA vaccination resulted in an OOI of <2 weeks with a 100% protection. Challenge virus titres were reduced by 2 Log 10 at 2 weeks p.v. and >4 Log 10 at 3 weeks p.v. The 100% protection correlated well with the high HI titres and H5 ELISA titres at 2 and 3 weeks p.v. Surprisingly, some chickens had HI titres <2 but these were still protected for HPAI H5N1 challenge. Thus, the HI titres do not always correlate with protection.

The HVT-H5 HA stem vaccine performed less in SPF chickens compared to the HVT-Full H5 HA vaccine. At 3 weeks p.v. only 60% of chickens were protected. Also, the reduction in challenge virus replication in the trachea was only reduced by 1 Log 10.

In strong contrast to the good protection by the HVT-Full H5 HA vaccine in SPF animals, this vaccine performed very poorly in AIV MDA+ animals with protection of only 43% and 46% at 4 weeks and 5 weeks p.v., respectively. The poor protection also correlates with poor serological responses and marginal effects on challenge virus replication in the trachea. Clearly, the HVT-Full H5 HA vaccine is severely hampered by the high AIV H5 MDA levels.

Interestingly, although the HVT-H5 HA stem vaccine performed poorly in SPF animals, the vaccine elicited 86% and 93% protection at 4 weeks and 5 weeks p.v. in AIV MDA+ animals, respectively. Also, the HVT-H5 HA stem vaccine reduced virus titres in the trachea more efficiently compared to the HVT-Full H5 HA vaccine. The efficient protection also correlates with higher antibody titres on day of challenge in HVT-H5 HA stem vaccinated chickens.

In conclusion, the HVT-Full H5 HA vaccine has an onset of immunity (OOI) of <2 weeks in SPF animals but is strongly impaired by MDA titres. The HVT-H5 HA stem vaccine has an OOI of >3 weeks in SPF animals but elicits high level of protection in the context of Influenza MDA at 4 and 5 weeks. Thus, vaccination with an antigen that is an HA stem polypeptide of the invention, is not affected by pre-existing influenza antibodies.

### Example 3: Tests in Chickens without Pre-Existing Antibodies

The experiments as described in Example 2 above, succeeded the initial experiments by the inventors which were done in SPF chickens, and which gave some partially disappointing results. In an experiment of the same general set-up and performance as that described in Example 2 above, the protective efficacy of different HA stem polypeptides was tested against a severe Influenza virus challenge infection at 3 weeks p.v., using a heterologous AIV H5N1 strain. As initial experiment this was done in target animals without pre-existing influenza antibodies, namely in SPF chickens. The different types of HA stem polypeptide vaccines tested were: a purified subunit, two types of an HVT vector vaccine, and an RP vaccine.

### 3.1. MATERIALS AND METHODS

Specifically the vaccines were prepared as follows:
the subunit vaccine contained an H5 HA stem polypeptide of the same amino acid composition as that in SEQ ID NO: 7, but only up to aa number 272, therefore it did not contain the TM and cytoplasmic domains of HA2. For purification purposes it contained a C-terminal Flag-tag/EK-cleavage site, followed by a triple Strep-tag. The subunit was expressed in HEK293T cells, purified, and was adjuvated with a standard liquid paraffin mineral oil, formulated as a water-in-oil emulsion. The subunit was administered at 4 μg/animal dose.
the HVT vector vaccines comprised either the H5 HA stem polypeptide of SEQ ID NO: 7, or contained the full H5 HA protein.
the RP was a VEEV-based RP comprising the H5 HA stem polypeptide of SEQ ID NO: 7. The RP was prepared and purified essentially as described. The RPs were administered at 1×10\hat{ }8 RP/animal dose, in aqueous buffer and adjuvated 1:1 with XSolve™ adjuvant (MSD Animal Health) as an emulsion.

One of the intentions of the inventors was to confirm the protective properties of an HA stem polypeptide as described in WO2013/079473 and the corresponding paper by Impagliazzo (supra). However this gave an unexpectedly poor result.

Like in Example 2, chickens were vaccinated at day old, by sc route, with 0.2 ml HVT-Full H5 HA, HVT-H5 HA stem, VEEV-H5 HA stem RPs, or with adjuvated HA Stem subunit vaccine. A group of 5 chicks remained untreated, and a group of 10 chicks served as non-vaccinated challenge controls. Serum samples were taken at T=0 (day of vaccination) and T=20 (one day prior to challenge) and used for HI test and H5-specific ELISA assays. At T=21 animals were challenged with a lethal dose of an HPAI AIV strain: A/duck/Biddinghuizen/NL/2016 (H5N8, Glade 2.3.4.4), and morbidity and mortality were followed for 10 days. To determine challenge virus replication, trachea swabs were taken and analysed using an Influenza virus M-gene specific RT-qPCR assay.

### 3.2. RESULTS

The results showed that all controls were as expected: all non-vaccinated challenged chicks had died or were moribund by day 2 from challenge, and the negative controls were seronegative in HI and ELISA. Further all chicks vaccinated with Full HA protein showed strong HI titres, and all vaccinates showed an ELISA titre above background values, indicating all vaccines had been properly administered.

Results of challenge-survival for the vaccinated groups at 10 days post challenge was:
HVT-Full H5 HA: 100%
HVT-H5 HA stem: 70%
VEEV-H5 HA stem RP: 80%
H5 HA stem soluble subunit: 0%

The HVT-Full H5 HA vaccine induced a robust seroconversion 3 weeks after vaccination. The strong serology correlated well with the 100% protection of this vaccine in SPF chickens against homologous as well as heterologous challenge. Interestingly, the full HA protein thus worked very well as vaccine in seronegative animals; quite contrary to the results found in Example 2, when applied to animals with pre-existing Influenza antibodies.

Surprisingly, the HA Stem subunit vaccine did not protect chickens from HPAI challenge; while mortality was delayed to some extent, all chicks in this group had died or were moribund by day 4 p.c., This result was in stark contrast to the positive reports as published.

However, when the HA Stem polypeptide was provided with a TM domain, there was a significant level of protection, both when delivered by a viral vector, and when delivered as an RP.

The H5N8 challenge strain used, was heterologous to the vaccine as it had 91.7% aa sequence identity.

### 3.2.1. Serology

A commercial inhibition ELISA test kit (AIV-H5 ELISA, IDVet) was used according to the manufacturer's instructions, to test the serum collected from chicks during the experiment.

Serum from SPF animals prior to vaccination (hatch mates) showed only 4% inhibition, which is within background level, the same applies to the titres of the unvaccinated controls at 3 weeks p.v., which showed only 2% inhibition, demonstrating the total absence of anti-H5 HA antibodies in the SPF chicks.

After vaccination of SPF animals with the HVT-Full H5 HA vaccine, inhibitory titres of 58% were found at 3 weeks p.v. This correlates very well with the results presented in Example 2.

Similarly, vaccination of SPF chicks with vector vaccines expressing the HA stem polypeptide according to the invention, resulted in inhibitory titres of 32% for the HVT-H5 HA stem vaccine, and of 28% for the VEEV-H5 HA stem RP vaccine. Both these results match those found in Example 2.

Finally, vaccination of chicks with soluble HA stem antigen as subunit vaccine, induced titres with only 9% inhibition in the ELISA.

These ELISA titres match very well with the challenge-survival results found in this experiment. Also, they show that expression of the HA stem polypeptide for the invention, as a membrane-associated antigen (thus with a TM-domain) and via a recombinant vector vaccine, induces more potent immune responses compared to a subunit vaccine based on soluble HA stem antigen.

### Example 4: Extended Tests of Challenge Protection

### 4.1. INTRODUCTION

In a further experiment like those of Examples 2 and 3 above, the protection against an HPAI H5N1 AIV challenge virus was tested again, in chickens with- and without pre-existing Influenza antibodies. However, to determine the efficacy of the vaccine under more “field-like” conditions, a contact challenge model was used in which vaccinated animals were challenge-infected and housed together with vaccinated non-challenged sentinel animals. As a control, also non-vaccinated animals were challenged and housed together with non-vaccinated non-challenged sentinels. Challenge virus replication was measured via trachea swabs, and challenge virus shedding was tested via cloaca swabs.

HVT-Full H5 HA and HVT-H5 HA stem vaccines were evaluated in one-day-old chickens that were either SPF or AIV H5 HA MDA+ (offspring of vaccinated SPFs, as described above). Half of the vaccinated or non-vaccinated control SPF animals were subjected to a challenge with H5N1 challenge virus 5 weeks p.v. Half of the AIV MDA+ vaccinated or non-vaccinated animals were subjected to a similar challenge, but at 6 weeks p.v. Eight hours post challenge sentinel birds were added to the group of directly challenged animals. Serological responses, mortality and challenge virus replication in the trachea and virus shedding in the cloaca were assessed.

Similar protection levels were found as described in Examples 1 and 2, with also good reduction of challenge virus transmission by a vaccine for use according to the invention.

### 4.2. SET-UP

Control blood samples were taken before the start of the experiment from 5 SPF and 10 MDA+ chicken hatch mates, to determine day 0 antibody status. Excluding controls, both the SPF and the MDA+ groups had 76 chicks, these were in turn divided into separate groups divided over the various treatments: non-vaccinated/Full HA/HA stem vaccines; challenge/no challenge; and sentinel/test animal. At the lowest level each test group had 6 chicks. Vaccines were administered at day 1 to the intended groups, at about 2,000 PFU/dose in 0.2 ml, by sc route. Just before challenge, at days 35 (SPF) and 42 (MDA+), blood samples were taken of the respective animals for HI testing. Challenge virus was HPAI H5N1 A/turkey/Turkey/01/05 (clade 2.2.1); the challenge dose administered was determined to be 10\hat{ }3.6 respectively 10\hat{ }3.2 TCID50/animal. Challenged animals were not combined with sentinels until 8 hours after challenge. Cloaca swab samples were collected on day 1, 2, 3, 4, 6, and 8 p.c. from challenged chickens. Samples were collected using cotton swabs (dry rayon tip, Copan 155C). Directly following sampling, the swabs were agitated in approximately 2 mL of tryptose phosphate buffer supplemented with antibiotics and kept on melting ice during transport to the lab. The experiment ended 2 weeks after challenge, at day 49, respectively day 56.

### 4.3. RESULTS

### 4.3.1. Mortality

The results once again showed all controls to be as expected: all non-vaccinated challenged chicks had died or were moribund by day 2 from challenge, and the negative controls were seronegative in HI and ELISA. Non-vaccinated challenged MDA+ chicks died or had to be euthanized between day 3 and 6 post challenge with a mean death time of 4.3 days. MDA-level thus did elicit some protection but not enough to survive the lethal challenge. All chicks vaccinated with Full HA protein showed strong HI titres, and all vaccinates showed an ELISA titre above background values, indicating all vaccines had been properly administered.

All SPF sentinel animals that were non-vaccinated and in contact with the challenged animals died or had to be euthanized starting from day 3 until day 7 post challenge. Also, all non-vaccinated MDA+ sentinel animals in contact with the challenged animals died or had to be euthanized starting from day 6 until day 11 post challenge. Consequently, the transmission of the H5N1 TT05 virus from challenged to sentinel animals was very robust.

### Results of Test Groups

SPF animals—vaccinated: Survival Reduction of transmission to sentinels

Directly challenged:

| | | |
|---|---|---|
| HVT-Full H5 HA: | 100% | 89% |
| HVT-H5 HA stem: | 72% | 72% |

Sentinels:

| | | |
|---|---|---|
| HVT-Full H5 HA: | 100% | — |
| HVT-H5 HA stem: | 100% | — |

MDA+ animals—vaccinated:

Directly challenged:

| | | |
|---|---|---|
| HVT-Full H5 HA: | 22% | 56% |
| HVT-H5 HA stem: | 83% | 89% |

Sentinels:

| | | |
|---|---|---|
| HVT-Full H5 HA: | 78% | — |
| HVT-H5 HA stem: | 100% | — |

In SPF animals, The HVT-Full H5 HA vaccination resulted in 100% protection of challenged animals, at 5 weeks p.v. This result confirmed and fully matched those found in the previous trials.

However, and again, the HVT-Full H5 HA vaccine in MDA+ animals, challenged at 6 weeks p.v., could only protect 22% of the animals from death. This was even worse as the 43% and 46% protection that was measured at 4 and 5 weeks p.v. in Example 2.

The protection provided by the HVT-H5 HA stem vaccine against the severe challenge was very effective, giving 83% survival at 6 weeks p.v. These results show that a vaccine comprising the HA stem polypeptide according to the invention is not hampered by high levels of pre-existing Influenza HA antibodies.

### 4.3.2. Reduction of Challenge Virus Transmission

The challenge virus was efficiently transmitted from non-vaccinated SPF and MDA+ animals to non-vaccinated sentinel SPF and MDA+ animals. Vaccination of SPF animals with the HVT-Full H5 HA vaccine reduced transmission of the challenge virus to non-detectable levels. In contrast, vaccination of MDA+ animals with the HVT-Full H5 HA vaccine only reduced transmission to some extent but could not prevent transmission from challenged to sentinel animals, with an even mortality as consequence.

The HVT-H5 HA stem vaccine was able to protect the majority of the SPF and MDA+ chickens from challenge mortality. In addition, in both SPF as well as MDA+ animals the transmission of challenge virus was fully blocked. Thus, the use of the HA stem polypeptide according to the invention in a population having pre-existing Influenza antibodies will protect most targets from clinical disease, and cuts off viral transmission completely.

The results of virus replication in the vaccinated and sentinel animals showed similar patterns, with the HVT-Full H5 HA vaccine being effective in SPF but not in MDA+ animals, with the reversed result for the HVT-H5 HA stem vaccine.

### 4.3.3. Serology

HI titres in MDA+ hatch mates at day 0 were 159, which was considerably higher than those in the experiment described in Example 2.

A commercial inhibition ELISA test kit (AIV-H5 ELISA, IDVet) was used according to the manufacturer's instructions, to test the serum samples obtained from chicks during the experiment.

Serum from SPF animals prior to vaccination (hatch mates) showed an inhibition of 5% and non-vaccinated SPF animals (negative controls) showed an inhibition of 6%, both at background levels.

Vaccination of SPF animals with the HVT-Full H5 HA vaccine resulted in inhibitory titres of 82% at 5 weeks p.v. In contrast to these high antibody titres in SPF animals, AIV-H5 MDA+ animals vaccinated with the HVT-Full H5 HA vaccine produced sera with only 38% inhibition at 6 weeks p.v. These H5 antibody titres were only slightly higher than the 26% observed in the non-vaccinated MDA+ animals, which shows that the HVT-Full H5 HA vaccine was severely hampered by the pre-existing AIV-H5 antibody titres at the day of vaccination.

Vaccination of SPF animals with the HVT-H5 HA stem vaccine resulted in inhibitory titres of 48% at 5 weeks p.v. Surprisingly, vaccination of AIV-H5 MDA+ animals with the HVT-H5 HA stem vaccine resulted in sera with 53% inhibition at 6 weeks p.v. These H5 antibody titres were higher compared to those in HVT-H5 HA stem vaccine SPF animals at 5 weeks p.v., and much higher compared to those in the non-vaccinated MDA+ animals. Thus, the HVT-H5 HA stem vaccine is not affected by pre-existing H5 antibodies at the time of vaccination.

### 4.4. CONCLUSIONS

HVT-Full H5 HA and HVT-H5 HA stem vaccines were evaluated in one-day-old SPF- and in AIV MDA+ chickens. Half of the vaccinated or non-vaccinated control SPF animals were subjected to a challenge with HPAI H5N1 virus at 5 weeks p.v. Half of the vaccinated or non-vaccinated AIV MDA+ animals were subjected to the challenge infection at 6 weeks p.v. Eight hours post challenge the other half of animals were added to the group to determine challenge virus transmission from directly challenged animals to sentinel animals. Serological responses, mortality and challenge virus replication in the trachea and virus shedding in the cloaca were assessed.

HVT-Full H5 HA vaccination of SPF animals resulted in seroconversion of 97% of the animals and a 100% protection against challenge. Challenge virus replication in the oral cavity was strongly reduced by 2-3 Log 10. Shedding of viral RNA was also strongly reduced to nearly undetectable levels. Transmission from directly challenged birds to sentinel birds was reduced by 89%, based on RT-qPCR results, but all sentinel birds were protected from clinical disease.

HVT-Full H5 HA vaccination of AIV H5 MDA+ animals did not induce detectable HI titres 6 weeks p.v. The poor seroconversion correlated with a poor protection of 22% after challenge. Challenge virus replication in the oral cavity was only reduced by about 1 Log 110, although viral RNA shedding from the cloaca was significantly reduced. However, the reduction did not prevent transmission from directly challenged to sentinel animals, with a reduction in transmission rate of only 56% based on RT-qPCR results.

HVT-H5 HA stem vaccination of SPF animals reduced challenge virus replication in the oral cavity by 2 Log 10, which was only slightly less efficient as for the HVT-Full H5 HA vaccine. This correlated with the slightly reduced protection of 72% for lethal challenge. Shedding of viral RNA from the cloaca from challenged animals was strongly reduced by about 3 Log 10, and consequently the transmission from directly challenged birds to sentinel birds was reduced by 72% based on RT-qPCR results.

HVT-H5 HA stem vaccination of AIV H5 MDA+ animals resulted in 83% protection 6 weeks p.v., while only 22% of the birds were protected after vaccination with the HVT-Full H5 HA vaccine. Moreover, HVT-H5 HA stem vaccination of AIV H5 MDA+ animals reduced transmission by 89% based on RT-qPCR results.

In conclusion: the HVT-Full H5 HA vaccine is very potent in protecting SPF chickens for lethal H5N1 challenge while transmission is efficiently blocked. In AIV MDA+ animals, however, the protection of the HVT-Full H5 HA vaccine was only 22% and there was also sustainable transmission between directly challenged and sentinel animals with a mortality rate of 22%. This shows that the HVT-Full H5 HA vaccine is strongly affected by AIV H5 MDA titres. As observed before, the HVT-H5 HA stem vaccine elicits moderate protection levels in SPF animals. However, in AIV H5 MDA+ animals the HVT-H5 HA stem vaccine results in 83% protection and blocks transmission to sentinel animals very efficiently. Thus, a vaccine based on an HA stem polypeptide according to the invention, seems not to be affected by pre-existing Influenza HA antibodies, and therefore this antigen can be a solution to fill up the immunological gap of vaccine based on a Full HA protein, when applied in MDA+ targets.

### Example 5: Test of HA Stem-Combination Vaccine

In a follow-on experiment one of the things tested was whether the combined vaccination with a HA stem-polypeptide of the invention and a full HA protein could improve the immune-protection provided by each of these separately. The set-up of the experiment was largely as described in Examples 2-4 above, using AIV H5 HA MDA+ chicks.

The combined vaccination applied was by administration of 2000 pfu/dose of HVT-Full H5 HA in 0.2 ml by s.c. route in the neck, and of 1×10\hat{ }8/dose of a VEEV RP of H5 HA stem polypeptide in 0.2 ml of an O/W emulsion with XSolve by i.m. route in a leg. Each vaccine was essentially as described in Example 3 above. A group of 40 AIV H5 HA MDA+ layer chicks received the combined vaccination at day 1 of age. A group of 25 chicks served as unvaccinated MDA+ controls. Blood samples were taken at various times during the experiment, up to 8 weeks post vaccination.

### 5.1. Serology by ELISA

The commercial inhibition ELISA test kit (AIV-H5 ELISA, IDVet) was used to test the serum samples obtained from chicks throughout the experiment.

Serum from MDA+ hatch mates showed high H5 antibody titres with an average inhibition of 89%. These MDA titres decreased in non-immunized animals to 22% and 8% at 6 and 7-8 weeks, respectively.

The combination vaccine induced very high anti-H5 HA antibody titres in the MDA+ animals, scoring >65% inhibition in all chicks and at all time points tested at 6, 7, and 8 weeks p.v. Such high levels of antibodies correlate with a 100% protection against even a heterologous H5 strain influenza infection.

H5 antibody titres of such high levels had not been achieved by the inventors before after vaccination of AIV H5 HA MDA+ animals; not with a HA stem polypeptide vaccine, and certainly not with an HVT-Full H5 HA vaccine.

### Example 6: Tests in Swine

### 6.1. Efficacy of SIV H1 RP Vaccine in MDA− and MDA+ Piglets.

To test the effect of MDA on full length HA in swine, an RP vaccine expressing swine influenza virus (SIV) H1 HA protein was administered to young pigs, both MDA negative and -positive for H1 SIV.

To generate MDA+ piglets, high health sows seronegative for SIV were vaccinated twice during pregnancy, with a gamma-ray inactivated vaccine of SIV pandemic H1N1 virus: A/swine/Minnesota/A01483170/2014, which was formulated with XSolve adjuvant into an O/W emulsion. The sow vaccine had 10\hat{ }6 TCID50 equivalent per ml.

Two weeks after birth, blood was taken from all healthy piglets from the vaccinated sows. SIV MDA titres were determined using a standard HI protocol. Based on these results, mixed groups of 10 piglets were formed, having the same group-average MDA titre of: 6.6 Log 2 HI, and with equal division of individual MDA titres between 4 and 8 Log 2 HI.

Two groups of piglets, one MDA−, one MDA+, received a PBS mock vaccine. Two more groups, one MDA− and one MDA+, received a VEEV-RP vaccine expressing the full length H1 HA protein of SIV strain: A/swine/England/10/2010 (H1N1)) (GenBank: AFR75956); which has 97.5% amino acid sequence identity. The RPs were formulated into an O/W emulsion with XSolve adjuvant.

Piglets were vaccinated twice: a prime vaccination at 5 weeks of age (experimental day 1), and a booster at 8 weeks of age (exp. day 21). The RP vaccine was administered at 5×10\hat{ }6 particles per animal dose, given in 1 ml, intramuscularly in the neck.

At several times during the experiment blood samples were taken, for serum isolation. The results of the HI titre determinations are represented in FIG. 1.

The non-vaccinated MDA− control animals were and remained HI negative throughout the experiment. In the non-vaccinated MDA+ control animals, the MDA titres declined from about 6.6 Log 2 HI at day 14 of age, to background levels at about day 65 of age, and remained that way till the end of the experiment (T=56).

In the vaccinated piglets, the MDA− animals showed a small increase in HI titre after the prime vaccination, but a very strong increase after the booster. The group-average titre reached at 9 days post booster was 10.2 Log 2 HI. On the other hand, in the MDA+ piglets the HI titre initially dropped somewhat after prime vaccination, and after booster it did increase, but only to a group-average titre of 6.8, much lower as compared to that reached in the MDA− pigs.

This demonstrates that—like in chickens—in swine the efficacy of a vaccination with full HA protein is also severely hampered by the presence of pre-existing anti-HA head antibodies at the time of vaccination.

6.2. Experiments with H1-HA Stem Polypeptides

To confirm that vaccination with HA stem polypeptides according to the invention can overcome the effect of pre-existing HA antibodies, and to compare the different platforms for delivery and expression, further vaccination-challenge experiments in swine are in preparation. Both MDA− and MDA+ piglets will be vaccinated with an H1 HA stem polypeptide and with full H1 HA, and challenged with a live H1 SIV. Vaccines will be DNA plasmid expressed VEEV replicon RNAs, and VEEV RPs, with an oily adjuvant.

SIV H1 MDA+ piglets will be prepared and grouped as described in section 6.1. Both MDA− and MDA+ pigs will receive two vaccinations: at 5 and at 8 weeks of age. Main test vaccines will be: a pVAX plasmid delivered VEEV replicon RNA of either full H1 HA, or of H1 HA stem polypeptide as described in SEQ ID NO: 4. Control will be the RP vaccine of full H1 HA, as described in section 6.1 above, which is expected to perform poorly in MDA+ targets.

RP vaccines will be 5×10\hat{ }6 particles per animal dose, and plasmid vaccines will be 50 μg/animal dose. Both vaccine types will be formulated into an O/W emulsion with XSolve adjuvant, and will be administered in 2 ml intramuscularly in the neck.

Challenge infection will be given at about 11 weeks of age. The challenge virus will be: A/swine/Minnesota/A01483170/2014 (H1N1pdm), which will be administered at 1×10\hat{ }6 TCID50 per animal, in 5 ml of PBS (10 mM), intra-tracheally, and the necessary containment measures will be applied at BSL 2 level.

To monitor challenge virus replication, nasal swabs will be taken from all pigs before, and for 3 days from challenge. Observations will be made for clinical signs of infection such as anorexia, shortness of breath (dyspnoea), fever, cough and nasal discharge.

At 3 days post challenge, all animals will be sedated and bled for post-mortem investigation of lung lesions. Next to macroscopic scoring, samples of lung tissue will be taken for challenge virus quantification.

### Example 7: Test of Expression in Host Cells

To investigate the expression of HA stem polypeptides in host cells, a series of experiments were performed using different forms for the delivery of the polypeptide according to the invention to host cells. Different staining techniques were applied to visualise the type and the location of those expressions.

### 7.1. Hela Cells

In one approach, Hela R19 cells were transfected with HA stem expressing plasmids, in short: cells were seeded in 96 well plates and cultured overnight to reach about 80% confluency. Next day a transfection mixture was prepared with per well: 0.1 μg of plasmid DNA, 0.3 μl FuGENEHD® (Promega) (non-lipid) transfection reagent, and 4.6 μl OptiMEM® medium (ThermoFisher), this was incubated for 15 minutes at room temperature. Next this was added to the cells in DMEM+10% v/v serum but no antibiotics, and incubated overnight. After 24 hours the cells were fixed with 3.7% formaldehyde containing 1% methanol. This type of fixation assures the cell-membranes are still intact, so that any signal observed must be cell-surface expressed. Cells were stained with FI6 (human anti-HA stem) antibody, and with a secondary Goat anti-human IgG Alexa488 antibody (Molecular probes, Thermo fisher) in a standard IFT protocol.

The results showed that both the H1 HA stem- and the H9 HA stem polypeptides were clearly detectable at the cell surface of HeLa cells, while mock transfected cells remained negative. This shows that the HA stem polypeptides are properly expressed, and are displayed on the surface of host cells transfected with a vector according to the invention.

### 7.2. Vero and CHO Cells

In a similar series of experiments, Vero Ames and CHO-K1 cells were transfected with plasmids expressing the H1- or H9 HA stem polypeptides of the invention. After transfection and incubation, cells were fixed with 4% formaldehyde in phosphate-buffered saline (PBS) and incubated for 15 minutes at room temperature. This type of fixation assures the cells are still intact, so that any signal observed must be cell surface exposed. Some cells were additionally treated with PBS containing 0.1% Triton-×100, which permeabilises the cells to allow both intra- and extracellular staining of the antigens. Next, IFT assay using FI6 as primary antibody was performed.

The results showed that while mock transfected cells remained negative, both in Vero and in CHO cells the H1- and H9 HA stem polypeptides were well expressed. In the permeabilized cells, staining was seen all over the cells; also without permeabilization, the polypeptides could be clearly detected by the FI6 antibody. This demonstrates an efficient expression of these polypeptides in CHO and in Vero host cells. In addition this shows there was presentation on the cell-surface of the H1 and H9 HA stem polypeptides, also in these types of host cells.

In a follow-on experiment, Vero cells were transfected with plasmids expressing the H1-, H5-, and H9 HA stem polypeptides of the invention, or with plasmids expressing Full HA protein of H1, H5, or H9.

After transfection and incubation, the cells were fixed with formalin/PBS to keep them intact and stained in an IFT assay with FI6 as first antibody.

Results showed that (while mock cells were negative) Vero cells expressing either full HA protein or HA stem polypeptide were all showing clear staining on the cell surface. Consequently, the H1-, H5- and H9 HA stem polypeptides of the invention are displayed on the cell surface, in the same way as the full HA proteins are.

### Example 8: Tests with H9 HA Stem Polypeptides

To test further variants of Influenza virus, and demonstrate that different vector types can be used, experiments are in preparation with H9 HA: both full HA protein and HA stem polypeptide will be tested in chickens, both SPF and H9 MDA+, and both as plasmid delivered replicon RNA molecules, and as RPs. Both the plasmid delivered replicon RNA and the RPs will provide for expression of the H9 HA stem polypeptide of SEQ ID NO: 12, which is a consensus sequence of recent H9 AIV isolates, and contains the modifications as described herein: a deletion of the head domain, which was replaced by a 4× G linker, and contains a trimerization-, TM- and a cytoplasmic domain. The encoding nucleotide (SEQ ID NO: 11) was codon-optimised towards the transcription profile of chickens, and a number of stabilising mutations were applied.

The H9 MDA+ chicks were generated by vaccination of SPF parents with an inactivated H9N2 AIV vaccine, of which the H9 amino acid sequence was 97% identical to that of the H9 HA stem polypeptide, thus providing for close-to homologous MDAs. Vaccinations will be given at day 1 of age.

Challenge will be applied at 4 weeks after vaccination for the SPF chickens, and at 5 weeks p.v. for the MDA+ animals. The challenge material is egg-grown allantoic fluid of the LPAI strain A/chicken/Egypt/V1527/2018 (H9N2), of which the H9 aa sequence is 94% identical to that of the used H9 HA stem polypeptide. This will be administered intranasally at 10\hat{ }6 EID50 per animal in 0.2 ml.

Swaps will be taken for the first week post challenge, and clinical signs will be monitored for two weeks post challenge.

To compare the effect of MDA on full length HA, vaccinations with a full length H9 HA of similar sequence will be done, using similar plasmid- and RP vaccines. In addition an HVT-H9 HA recombinant viral vector will be given subcutaneously to an additional group of chicks. This HVT vector contains the HA gene inserted into the HVT genome in between the UL44 and UL45 genes, and is driven by the PRV gB promoter.

The replicon RNA vaccines will be administered as plasmids, given intramuscularly at 10 μg per animal dose in 0.2 ml. One group will only receive 1 μg/dose to test the effect of plasmid dose. The plasmids will be formulated in polyacrylic polymer nanogel (20 Med Therapeutics) at 2.5 mg/ml in 20 mM HEPES+5% Trehalose. The RPs are VEEV RPs and will be administered intramuscularly in aqueous buffer at a dose of 10\hat{ }8 RP per animal in 0.2 ml in adjuvated emulsion.

Before, throughout, and at the end of the experiment blood samples will be taken from selected animals for monitoring the initial- and developing serological status. Also animals will be monitored, and clinical signs will be recorded. As the challenge strain is only LPAI, no mortalities or severe clinical signs are expected.

### LEGEND TO THE FIGURES

FIG. 1:

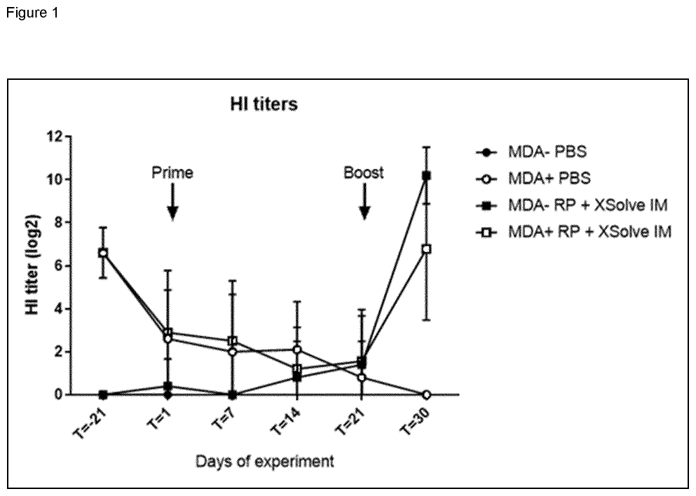

HI titre values (in Log 2) over time, from pigs vaccinated with an RP vaccine expressing full H1 HA. Piglets were MDA− or MDA+ for H1 HA at the start of the experiment. Details are described in Example 6.1.

1-7. (canceled)

8. A method for reducing infection or disease caused by an influenza virus in a target that has antibodies against an Influenza virus HA head domain at the time of vaccination, said method comprising administering to said target a vaccine comprising a recombinant vector capable of expressing a recombinant Influenza virus haemagglutinin (HA) stem polypeptide in the target and a pharmaceutically acceptable carrier; wherein said recombinant Influenza virus HA stem polypeptide comprises a headless Influenza virus HA stem domain, a trimerization domain, and a transmembrane domain.

9. The method of claim 8, wherein said recombinant Influenza virus HA stem polypeptide has the amino acid sequence selected from one of SEQ ID NO's: 4, 6, 8, 10, and 12.

10. The method of claim 9, wherein said recombinant vector is selected from a nucleic acid, a virus, and a replicon particle (RP).

11. The method of claim 8, wherein said recombinant vector is selected from a nucleic acid, a virus, and a replicon particle (RP).

12. The recombinant vector of claim 11, wherein:
the nucleic acid is a eukaryotic expression plasmid or an RNA molecule;
the virus is selected from a Herpesvirus, a Poxvirus, a Retrovirus, a Paramyxovirus, a Rhabdovirus and an Adenovirus; or
the RP is an Alphavirus RP.

13. The recombinant vector of claim 10, wherein:
the nucleic acid is a eukaryotic expression plasmid or an RNA molecule;
the virus is selected from a Herpesvirus, a Poxvirus, a Retrovirus, a Paramyxovirus, a Rhabdovirus and an Adenovirus; or
the RP is an Alphavirus RP.

14. A method for reducing infection or disease caused by an influenza virus in a target that has antibodies against an Influenza virus HA head domain at the time of vaccination, said method comprising administering to said target a host cell comprising a recombinant vector, said recombinant vector being capable of expressing a recombinant Influenza virus haemagglutinin (HA) stem polypeptide in the target; wherein said recombinant Influenza virus HA stem polypeptide comprises a headless Influenza virus HA stem domain, a trimerization domain, and a transmembrane domain.

15. The method of claim 14, wherein said recombinant Influenza virus HA stem polypeptide has the amino acid sequence selected from one of SEQ ID NO's: 4, 6, 8, 10, and 12.

16. The method of claim 15, wherein said recombinant vector is selected from a nucleic acid, a virus, and a replicon particle (RP).

17. The method of claim 14, wherein said recombinant vector is selected from a nucleic acid, a virus, and a replicon particle (RP).

18. The recombinant vector of claim 17, wherein:
the nucleic acid is a eukaryotic expression plasmid or an RNA molecule;
the virus is selected from a Herpesvirus, a Poxvirus, a Retrovirus, a Paramyxovirus, a Rhabdovirus and an Adenovirus; or
the RP is an Alphavirus RP.

19. The recombinant vector of claim 16, wherein:
the nucleic acid is a eukaryotic expression plasmid or an RNA molecule;
the virus is selected from a Herpesvirus, a Poxvirus, a Retrovirus, a Paramyxovirus, a Rhabdovirus and an Adenovirus; or
the RP is an Alphavirus RP.
